(19) United States
(12) Patent Application Publication
(10) Pub. No.: US 2005/0085477 A1
(43) Pub. Date: Apr. 21, 2005

(54) Compositions of a cyclooxygenase-2 selective inhibitor and a serotonin-modulating agent for the treatment of neoplasia

(75) Inventor: Jaime L. Masferrer, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/922,511

(22) Filed: Aug. 20, 2004

Related U.S. Application Data
(60) Provisional application No. 60/497,202, filed on Aug. 22, 2003.

(51) Int. Cl. A61K 31/50; A61K 31/415; A61K 31/18; A61K 31/365

(52) U.S. Cl. 514/247; 514/406; 514/473; 514/602

(57) ABSTRACT

The present invention provides compositions and methods for the treatment of neoplasia in a subject. More particularly, the invention provides a combination therapy for the treatment of a neoplasia comprising the administration to a subject of a serotonin modulating agent in combination with a cyclooxygenase-2 selective inhibitor.

### CROSS REFERENCE TO RELATED APPLICATION

1 This application claims priority from Provisional Application Ser. No. 60/497,202, filed on Aug. 22, 2003, which is hereby incorporated by reference in its entirety.

### FIELD OF THE INVENTION

The present invention provides compositions and methods for the treatment of a neoplasia. More particularly, the invention is directed toward a combination therapy for the treatment or prevention of neoplasia comprising the administration to a subject of a serotonin modulating agent and a cyclooxygenase-2 selective inhibitor.

### BACKGROUND OF THE INVENTION

Currently, non-surgical cancer treatment regimes involve administering one or more highly toxic chemotherapeutics or hormonal therapies to the patient after the cancer has progressed to a point where the therapeutic benefits of chemotherapy/hormonal therapies outweigh its serious side effects. As a consequence of these side effects, standard chemotherapeutics are typically used only for short periods of time, often alternating chemotherapy with periods off treatment, so as not to overwhelm the patient with drug side effects. Accordingly, given the risk-benefit trade-off, side effects typically preclude starting chemotherapy when patients exhibit precancerous lesions, or continuing chemotherapy or hormonal therapy on a chronic basis after cancer has been eliminated in an attempt to prevent its re-occurrence.

Cancer and precancer research is replete with publications that describe various biochemical molecules that are over-expressed in neoplastic tissue, leading several groups to research whether specific over-expressed molecules are responsible for the disease, and whether, if such over-expression were inhibited, neoplasia could be alleviated. One such biochemical molecule that has been extensively studied as a therapeutic target for neoplasia treatment is the prostaglandins, which are naturally occurring C-20 unsaturated fatty acids. By way of example, in familial adenomatous polyposis (“FAP”), Waddell et al. hypothesized that since prostaglandins were over-expressed in such polyps, non-steroidal anti-inflammatory drugs (“NSAIDs”) should alleviate the condition because NSAIDs inhibited prostaglandin synthesis. Thus, he administered the NSAID sulindac (an inhibitor of PGE₂) to several FAP patients. Waddell et al. discovered that polyps regressed and did not recur upon therapeutic treatment with an NSAID. PGE₂ inhibition results from the inhibition of cyclooxygenase (COX) by NSAIDs.

While patients treated with NSAIDS typically exhibit far fewer side effects than with conventional chemotherapeutics or hormonals, the use of high doses of most common NSAIDs can produce severe side effects, including life-threatening ulcers that limit their therapeutic potential. One reason proposed for the severe side effects associated with traditional NSAIDs is their non-selective inhibition of both of the cyclooxygenase enzymes (COX), commonly known as COX-1 and COX-2. COX-1 is constitutively expressed and mediates a number of physiological functions, such as kidney and gastrointestinal function. COX-2 expression, contrastingly, is stimulated by a number of inflammatory cytokines, growth factors, oncogenes, lipopolysaccharides, and tumor promoters. While conventional NSAIDs block both forms of the enzyme, a new class of NSAID, selective cyclooxygenase-2 inhibitors, provide a viable target of inhibition that more effectively reduces inflammation and produces fewer and less drastic side effects.

COX-2 plays a key role in tumorigenesis through stimulating epithelial cell proliferation, inhibiting apoptosis, stimulating angiogenesis, enhancing cell invasiveness, mediating immune suppression, and by increasing the production of mutagens. Results of several studies using mouse models of colon cancer and the results of clinical trials have shown COX-2 to be a useful target for the prevention and treatment of colon cancer (Fernandex et al., (2002) In Vivo 16(6):501-509). Studies with several other epithelial cancers involving different organ sites, e.g., breast, prostate, bladder, lung, and pancreas, suggest that COX-2 plays an important role in the pathogenesis of these cancers (e.g. for its role in breast cancer see Singh et al., (2002) J. Surg. Res. 108(1):173-179; for its role in fibroblasts and endothelial cells see Sonoshita et al., (2002) Cancer Res. 62(23):6846-6849; for its role in gastric cells see Li et al., (2002) 21(6):625-629).

Serotonin modulating agents such as selective serotonin reuptake inhibitors (SSRIs) are the treatment of choice for clinical depression and a range of anxiety-related disorders. In addition, studies indicate that a number of suitable serotonin modulating agents act directly on Burkitt lymphoma cells to trigger rapid and extensive programmed cell death (Serafeim et al., (2003) Blood April 15;101(8):3212-3219). Moreover, several studies have demonstrated a substantial reduction in nausea and vomiting for patients undergoing cancer chemotherapy that were also administered a serotonin modulating agent, such as a 5-hydroxytryptamine type 3 (5-HT3) receptor antagonist. (Tremblay et al., (2003) Clin Oncol June 1;21 (11):2147-2155).

### SUMMARY OF THE INVENTION

Among the several aspects of the invention is provided a method and a composition for the treatment of neoplasia in a subject. The composition comprises a cyclooxygenase-2 selective inhibitor or an isomer, ester, a pharmaceutically acceptable salt or a prodrug thereof and a serotonin modulating agent or an isomer, ester, a pharmaceutically acceptable salt or a prodrug thereof, and the method comprises administering to the subject a cyclooxygenase-2 selective inhibitor or an isomer, ester, a pharmaceutically acceptable salt or a prodrug thereof in combination with a serotonin modulating agent or an isomer, ester, a pharmaceutically acceptable salt or a prodrug thereof.

In one embodiment, the cyclooxygenase-2 selective inhibitor is a member of the chromene class of compounds. For example, the chromene compound may be a compound of the formula: 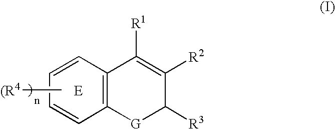
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In another embodiment, the cyclooxygenase-2 selective inhibitor or an isomer, ester, a pharmaceutically acceptable salt or a prodrug thereof comprises a compound of the formula: 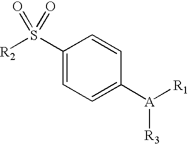
wherein
A is selected from the group consisting of partially unsaturated or unsaturated heterocyclyl rings and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl or amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In one embodiment, the serotonin modulating agent is a serotonin receptor antagonist.

In another embodiment, the serotonin modulating agent is a serotonin receptor agonist.

In another embodiment, the serotonin modulating agent is a serotonin reuptake inhibitor.

Other aspects of the invention are described in more detail below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl. The terms “alkenyl” and “lower alkenyl” also are radicals having “cis” and “trans” orientations, or alternatively, “E” and “Z” orientations.

The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH2.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to selectively inhibit cyclooxygenase-2 over cyclooxygenase-1. Typically, it includes compounds that have a cyclooxygenase-2 IC₅₀ of less than about 0.2 micro molar, and also have a selectivity ratio of cyclooxygenase-1 (COX-1) IC₅₀ to cyclooxygenase-2 (COX-2) IC₅₀ of at least about 5, more typically of at least about 50, and even more typically, of at least about 100. Moreover, the cyclooxygenase-2 selective inhibitors as described herein have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2-) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine(N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prevention” includes either preventing the onset of clinically evident neoplasia altogether or preventing the onset of a preclinically evident stage of neoplasia in individuals at risk. Also encompassed by this definition is the prevention of initiation for malignant cells or to arrest or reverse the progression of premalignant cells to malignant cells. This includes prophylactic treatment of those at risk of developing the neoplasia.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “serotonin modulating agent,” unless otherwise indicated herein, includes a number of suitable agents that alter serotonin levels by interacting with any of the serotonin receptor sites (5HT₁-5HT₇) in the body. The term also includes a number of suitable agents that either alter the biological activity of serotonin or result in a change in the amount of biologically active serotonin. Modulation can be an increase or decrease in activity, a change in binding characteristics, or any other change in the biological, functional, or immunological properties of biologically active serotonin.

The term “subject” for purposes of treatment includes any human or animal subject who has neoplasia. The subject can be a domestic livestock species, a laboratory animal species, a zoo animal or a companion animal. In one embodiment, the subject is a mammal. In another embodiment, the mammal is a human being.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of cyclooxygenase-2 selective inhibitor and the amount of serotonin modulating agent) which will achieve the goal of improvement in disorder severity and the frequency of incidence over no treatment or treatment of each agent by itself.

The term “treatment” includes partial or total inhibition of the neoplasia growth, spreading or metastasis, as well as partial or total destruction of the neoplasia cells. Treatment also includes prevention of a neoplasia or related disorder.

### DESCRIPTION OF THE PREFERRED EMBODIMENTS

The present invention provides a combination therapy comprising the administration to a subject of a therapeutically effective amount of a COX-2 selective inhibitor or an isomer, ester, pharmaceutically acceptable salt or a prodrug thereof and a therapeutically effective amount of a serotonin modulating agent or an isomer, ester, a pharmaceutically acceptable salt or a prodrug thereof. The combination therapy is used to treat neoplasias. When administered as part of a combination therapy, the COX-2 selective inhibitor together with the serotonin modulating agent provides enhanced treatment options as compared to administration of either the serotonin modulating agent or the COX-2 selective inhibitor alone.

### Cyclooxygenase-2 Selective Inhibitors

A number of suitable cyclooxygenase-2 selective inhibitors or isomers, pharmaceutically acceptable salts, esters, or prodrugs thereof, may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

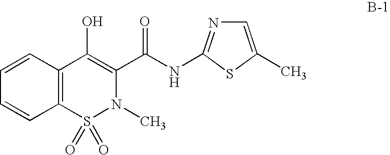

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

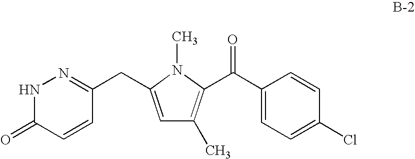

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having
Formula I shown below and possessing, by way of example and not limitation, the structures disclosed in Table 1. Furthermore, benzopyran cyclooxygenase-2 selective inhibitors useful in the practice of the present methods are described in U.S. Pat. Nos. 6,034,256 and 6,077,850 herein incorporated by reference in their entirety.

In another embodiment, the cyclooxygenase-2 selective inhibitor is a chromene compound represented by Formula I or an isomer, a pharmaceutically acceptable salt, ester, or prodrug thereof: 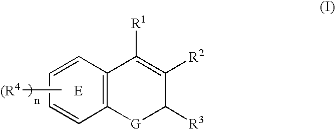
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, lower alkyl, lower aralkyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from the group consisting of alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,

wherein:

n is an integer which is 0, 1, 2, 3 or 4;
G is O, S or NR^{a};
R^{a} is alkyl;
R¹ is H;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In a further embodiment, the cyclooxygenase-2 selective inhibitor may also be a compound of Formula (I), or an isomer, a pharmaceutically acceptable salt, ester, or prodrug thereof,
wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,

wherein:

n is an integer which is 0, 1, 2, 3 or 4;
G is oxygen or sulfur;
R¹ is H;
R² is carboxyl;
R³ is lower haloalkyl; and
each R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,

wherein:

n is an integer which is 0, 1, 2, 3 or 4;
G is oxygen or sulfur;
R¹ is H;
R² is carboxyl;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof,

wherein:

n is an integer which is 0, 1, 2, 3 or 4;
G is oxygen or sulfur;
R¹ is H;
R² is carboxyl;
R³ is trifluoromethyl or pentafluoroethyl; and
each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof,

wherein:

n is 4;
G is O or S;
R¹ is H;
R²is CO₂H;
R³ is lower haloalkyl;
a first R⁴ corresponding to R⁹ is hydrido or halo;
a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;
a third R⁴ corresponding to R¹¹ is H, lower alkyl, halo, lower alkoxy, or aryl; and
a fourth R⁴ corresponding to R¹² is H, halo, lower alkyl, lower alkoxy, or aryl;
wherein Formula (I) is represented by Formula (Ia):

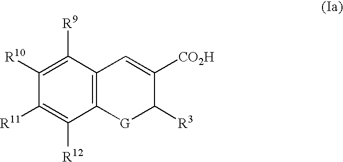

The cyclooxygenase-2 selective inhibitor used in connection with the method(s) of the present invention can also be a compound of having the structure of Formula (Ia) or an isomer, a pharmaceutically acceptable salt, ester, or prodrug thereof,

wherein:

G is O or S;
R³ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE I
EXAMPLES OF CHROMENE CYCLOOXYGENASE-2 SELECTIVE
INHIBITORS AS EMBODIMENTS

| Compound | |
|---|---|
| Number | Structural Formula |
| B-3 | 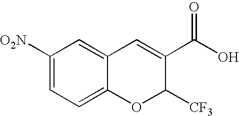 |
| | 6-Nitro-2-trifluoromethyl-2H-1- |
| | benzopyran-3-carboxylic acid |
| B-4 | 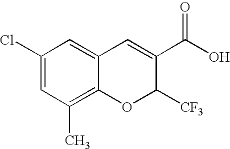 |
| | 6-Chloro-8-methyl-2-trifluoromethyl- |
| | 2H-1-benzopyran-3-carboxylic acid |
| B-5 | 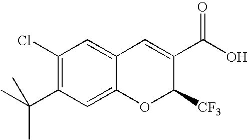 |
| | ((S)-6-Chloro-7-(1,1-dimethylethyl)-2-(trifluoro- |
| | methyl-2H-1-benzopyran-3-carboxylic acid |
| B-6 | 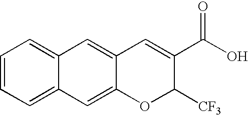 |
| | 2-Trifluoromethyl-2H-naphtho[2,3-b] |
| | pyran-3-carboxylic acid |
| B-7 | 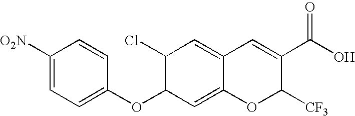 |
| | 6-chloro-7-(4-nitrophenaxy)-2-(trifluoramethyl)-2H-1- |
| | benzopyran-3-carboxylic acid |
| B-8 | 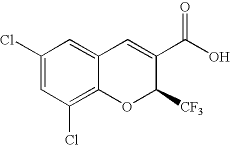 |
| | ((S)-6,8-Dichloro-2-(trifluoromethyl)- |
| | 2H-1-benzopyran-3-carboxylic acid |
| B-9 | 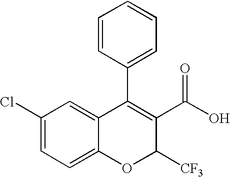 |
| | 6-Chloro-2-(trifluoromethyl)-4-phenyl-2H- |
| | l-benzopyran-3-carboxylic acid |
| B-10 | 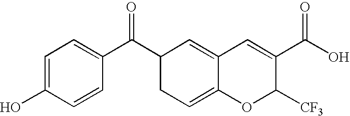 |
| | 6-(4-Hydroxybenzoyl)-2-(trifluoromethyl)- |
| | 2H-1-benzopyran-3-carboxylic acid |
| B-11 | 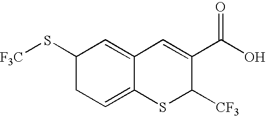 |
| | 2-(Trifluoromethyl)-6-[(trifluoromethyl)thiol]- |
| | 2H-1-benzothiopyran-3-carboxylic acid |
| B-12 | 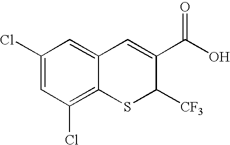 |
| | 6,8-Dichloro-2-trifluoromethyl-2H-1- |
| | benzothiopyran-3-carboxylic acid |
| B-13 | 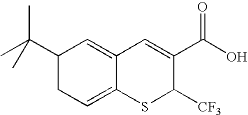 |
| | 6-(1,1-Dimethylethyl)-2-(trifluoromethyl)- |
| | 2H-1-benzothiopyran-3-carboxylic acid |
| B-14 | 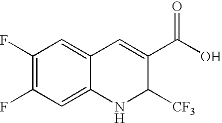 |
| | 6,7-Difluoro-1,2-dihydro-2-(trifluoro |
| | methyl)-3-quinolinecarboxylic acid |
| B-15 | 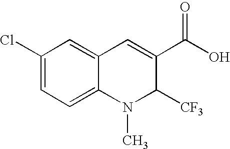 |
| | 6-Chloro-1,2-dihydro-1-methyl-2-(trifluoro |
| | methyl)-3-quinolinecarboxylic acid |
| B-16 | 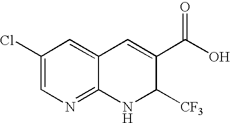 |
| | 6-Chloro-2-(trifluoromethyl)-1,2-dihydro |
| | [1,8]naphthyridine-3-carboxylic acid |
| B-17 | 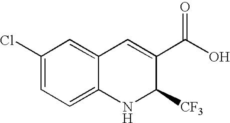 |
| | ((S)-6-Chloro-l, 2-dihydro-2-(trifluoro |
| | methyl)-3-quinolinecarboxylic acid |

In a further embodiment, the cyclooxygenase-2 selective inhibitor is selected from the class of tricyclic cyclooxygenase-2 selective inhibitors represented by the general structure of Formula II or an isomer, a pharmaceutically acceptable salt, ester, or prodrug thereof, 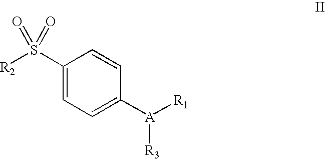
wherein:
A is selected from the group consisting of a partially unsaturated or unsaturated heterocyclyl ring and a partially unsaturated or unsaturated carbocyclic ring;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In another embodiment, the cyclooxygenase-2 selective inhibitor represented by the above Formula II is selected from the group of compounds illustrated in Table 2, consisting of celecoxib (B-18; U.S. Pat. No. 5,466,823; CAS No.16959042-5), valdecoxib (B-19; U.S. Pat. No. 5,633,272; CAS No.181695-72-7), deracoxib (B-20; U.S. Pat. No. 5,521,207; CAS No. 169590-41-4), rofecoxib (B-21; CAS No.162011-90-7), etoricoxib (MK-663; B-22; PCT publication WO 98/03484), tilmacoxib (JTE-522; B-23; CAS No. 180200-68-4), and cimicoxib (UR-8880; B23a; CAS No. 265114-23-6).

TABLE 2

| Compound | |
|---|---|
| Number | Structural Formula |
| B-18 | 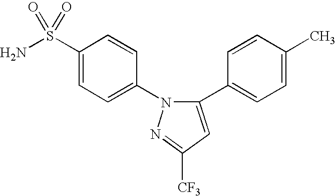 |
| B-19 | 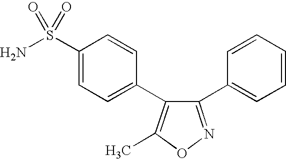 |
| B-20 | 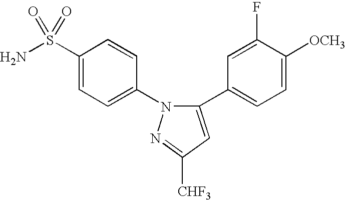 |
| B-21 | 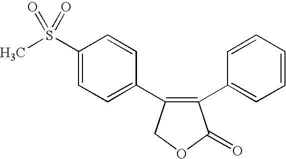 |
| B-22 | 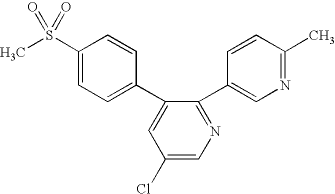 |
| B-23 | 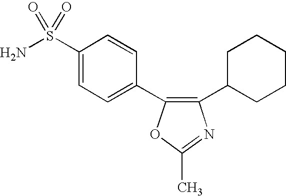 |
| B23a | 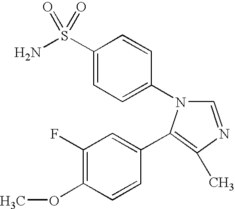 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No.198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

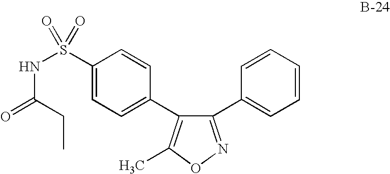

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

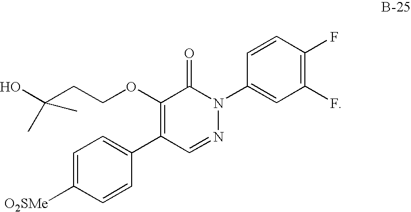

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

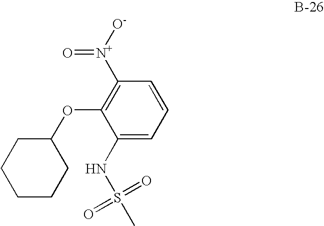

In yet a further embodiment, the cyclooxygenase-2 selective inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof: 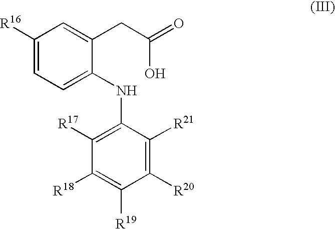
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided, however, that each of R¹⁷, R¹⁸, R²⁰ and R²¹ is not fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:
R¹⁶ is ethyl;
R¹⁷ and R¹⁹ are chloro;
R¹⁸ and R²⁰ are hydrogen; and
R²¹ is methyl.

In yet another embodiment, the cyclooxygenase-2 selective inhibitor is represented by Formula (IV) or an isomer, a pharmaceutically acceptable salt, ester, or prodrug thereof: 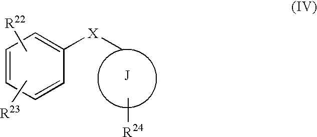
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

According to another embodiment, the cyclooxygenase-2 selective inhibitors or isomers, pharmaceutically acceptable salts, esters, or prodrugs thereof used in the present method(s) have the structural Formula (V) 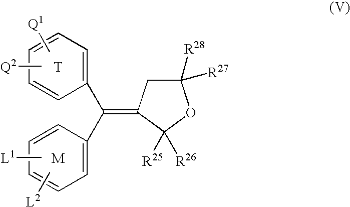
wherein:
T and M are independently phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
R²⁵ R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or
R²⁵ and R²⁶, together with the carbon atom to which they are attached, form a carbonyl or a saturated hydrocarbon ring having from 3 to 7 carbon atoms; or
R²⁷and R²⁸, together with the carbon atom to which they are attached, form a carbonyl or a saturated hydrocarbon ring having from 3 to 7 carbon atoms;
Q¹, Q², L¹ or L² are independently hydrogen, halogen, lower alkyl having from 1 to 6 carbon atoms, trifluoromethyl, lower methoxy having from 1 to 6 carbon atoms, alkylsulfinyl or alkylsulfonyl; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or Q¹ and Q² together form methylenedioxy; or L¹ and L² together form methylenedioxy.

In another embodiment, the compounds N-(2-cyclohexyloxynitrophenyl)methane sulfonamide, and (E)-4-[(4-methylphenyl)(tetrahydro-2-oxo-3-furanylidene)methyl]benzenesulfonamide or isomers, pharmaceutically acceptable salts, esters, or prodrugs thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3 below, include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1-methylethyl)-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-2-trifluoromethyl-2H-1-benzopyran-3-carboxylic acid (B-39);
7-(1-methylethyl)-2-trifluoromethyl-2H-1-benzopyran-3-carboxylic acid (B40);
7-phenyl-2-trifluoromethyl-2H-1-benzopyran-3-carboxylic acid (B-41);
6-chloro-7-ethyl-2-trifluoromethyl-2H-1-benzopyran-3-carboxylic acid (B42);
6-chloro-8-ethyl-2-trifluoromethyl-2H-1-benzopyran-3-carboxylic acid (B43);
6-chloro-7-phenyl-2-trifluoromethyl-2H-1-benzopyran-3-carboxylic acid (B44);
6,7-dichloro-2-trifluoromethyl-2H-1-benzopyran-3-carboxylic acid (B45);
6,8-dichloro-2-trifluoromethyl-2H-1-benzopyran-3-carboxylic acid (B46);
6-chloro-8-methyl-2-trifluoromethyl-2H-1-benzopyran-3-carboxylic acid (B47);
8-chloro-6-methyl-2-trifluoromethyl-2H-1-benzopyran-3-carboxylic acid (B48)
8-chloro-6-methoxy-2-trifluoromethyl-2H-1-benzopyran-3-carboxylic acid (B49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic acid (B-71);
7-(1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a)pyridine (B-75);
5,5-dimethyl-4-(4-methylsulfonyl)phenyl-3-phenyl-2-(5H)-furanone (B-76);
5-(4-fluorophenyl)-1-[4-(methylsulfonyl)phenyl]-3-(trifluoromethyl)pyrazole (B-77);
4-(4-fluorophenyl)-5-[4-(methylsulfonyl)phenyl]-1-phenyl-3-(trifluoromethyl)pyrazole (B-78);
4-(5-(4-chlorophenyl)-3-(4-methoxyphenyl)-1H-pyrazol-1-yl)benzenesulfonamide (B-79);
4-(3,5-bis(4-methylphenyl)-1H-pyrazol-1-yl)benzenesulfonamide (B-80);
4-(5-(4-chlorophenyl)-3-phenyl-1H-pyrazol-1-yl)benzenesulfonamide (B-81);
4-(3,5-bis(4-methoxyphenyl)-1H-pyrazol-1-yl)benzenesulfonamide (B-82);
4-(5-(4-chlorophenyl)-3-(4-methylphenyl)-1H-pyrazol-1-yl)benzenesulfonamide (B-83);
4-(5-(4-chlorophenyl)-3-(4-nitrophenyl)-1H-pyrazol-1-yl)benzenesulfonamide (B-84);
4-(5-(4-chlorophenyl)-3-(5-chloro-2-thienyl)-1H-pyrazol-1-yl)benzenesulfonamide (B-85);
4-(4-chloro-3,5-diphenyl-1H-pyrazol-1-yl)benzenesulfonamide (B-86);
4-[5-(4-chlorophenyl)-3-(trifluoromethyl)-1H-pyrazol-1-yl]benzenesulfonamide (B-87);
4-[5-phenyl-3-(trifluoromethyl)-1H-pyrazol-1-yl]benzenesulfonamide (B-88);
4-[5-(4-fluorophenyl)-3-(trifluoromethyl)-1H-pyrazol-1-yl]benzenesulfonamide (B-89);
4-[5-(4-methoxyphenyl)-3-(trifluoromethyl)-1H-pyrazol-1-yl]benzenesulfonamide (B-90);
4-[5-(4-chlorophenyl)-3-(difluoromethyl)-1H-pyrazol-1-yl]benzenesulfonamide (B-91);
4-[5-(4-methylphenyl)-3-(trifluoromethyl)-1H-pyrazol-1-yl]benzenesulfonamide (B-92);
4-[4-chloro-5-(4-chlorophenyl)-3-(trifluoromethyl)-1H-pyrazol-1-yl]benzenesulfonamide (B-93);
4-[3-(difluoromethyl)-5-(4-methylphenyl)-1H-pyrazol-1-yl]benzenesulfonamide (B-94);
4-[3-(difluoromethyl)-5-phenyl-1H-pyrazol-1-yl]benzenesulfonamide (B-95);
4-[3-(difluoromethyl)-5-(4-methoxyphenyl)-1H-pyrazol-1-yl]benzenesulfonamide (B-96);
4-[3-cyano-5-(4-fluorophenyl)-1H-pyrazol-1-yl]benzenesulfonamide (B-97);
4-[3-(difluoromethyl)-5-(3-fluoro-4-methoxyphenyl)-1H-pyrazol-1-yl]benzenesulfonamide (B-98);
4-[5-(3-fluoro-4-methoxyphenyl)-3-(trifluoromethyl)-1H-pyrazol-1-yl]benzenesulfonamide (B-99);
4-[4-chloro-5-phenyl-1H-pyrazol-1-yl]benzenesulfonamide (B-100);
4-[5-(4-chlorophenyl)-3-(hydroxymethyl)-1H-pyrazol-1-yl]benzenesulfonamide (B-101);
4-[5-(4-(N,N-dimethylamino)phenyl)-3-(trifluoromethyl)-1H-pyrazol-1-yl]benzenesulfonamide (B-102);
5-(4-fluorophenyl)-6-[4-(methylsulfonyl)phenyl]spiro[2.4]hept-5-ene (B-103);
4-[6-(4-fluorophenyl)spiro[2.4]hept-5-en-5-yl]benzenesulfonamide (B-104);
6-(4-fluorophenyl)-7-[4-(methylsulfonyl)phenyl]spiro[3.4]oct-6-ene (B-105);
5-(3-chloro-4-methoxyphenyl)-6-[4-(methylsulfonyl)phenyl]spiro[2.4]hept-5-ene (B-106);
4-[6-(3-chloro-4-methoxyphenyl)spiro[2.4]hept-5-en-5-yl]benzenesulfonamide (B-107);
5-(3,5-dichloro-4-methoxyphenyl)-6-[4-(methylsulfonyl)phenyl]spiro[2.4]hept-5-ene (B-108);
5-(3-chloro-4-fluorophenyl)6-[4-(methylsulfonyl)phenyl]spiro[2.4]hept-5-ene (B-109);
4-[6-(3,4-dichlorophenyl)spiro[2.4]hept-5-en-5-yl]benzenesulfonamide (B-110);
2-(3-chloro-4-fluorophenyl)-4-(4-fluorophenyl)-5-(4-methylsulfonyl phenyl)thiazole (B-111);
2-(2-chlorophenyl)-4-(4-fluorophenyl)-5-(4-methylsulfonyl phenyl)thiazole (B-112);
5-(4-fluorophenyl)-4-(4-methylsulfonylphenyl)-2-methylthiazole (B-113);
4-(4-fluorophenyl)-5-(4-methylsulfonylphenyl)-2-trifluoromethylthiazole (B-114);
4-(4-fluorophenyl)-5-(4-methylsulfonylphenyl)-2-(2-thienyl)thiazole (B-115);
4-(4-fluorophenyl)-5-(4-methylsulfonylphenyl)-2-benzylaminothiazole (B-116);
4-(4-fluorophenyl)-5-(4-methylsulfonylphenyl)-2-(1-propylamino)thiazole (B-117);
2-[(3,5-dichlorophenoxy)methyl)-4-(4-fluorophenyl)-5-[4-(methylsulfonyl)phenyl]thiazole (B-118);
5-(4-fluorophenyl)-4-(4-methylsulfonylphenyl)-2-trifluoromethylthiazole (B-119);
1-methylsulfonyl-4-[1,1-dimethyl-4-(4-fluorophenyl)cyclopenta-2,4-dien-3-yl]benzene (B-120);
4-[4-(4-fluorophenyl)-1,1-dimethylcyclopenta-2,4-dien-3-yl]benzenesulfonamide (B-121);
5-(4-fluorophenyl)-6-[4-(methylsulfonyl)phenyl]spiro[2.4]hepta-4,6-diene (B-122);
4-[6-(4-fluorophenyl)spiro[2.4]hepta-4,6-dien-5-yl]benzenesulfonamide (B-123);
6-(4-fluorophenyl)-2-methoxy-5-[4-(methylsulfonyl)phenyl]-pyridine-3-carbonitrile (B-124);
2-bromo-6-(4-fluorophenyl)-5-[4-(methylsulfonyl)phenyl]-pyridine-3-carbonitrile (B-125);
6-(4-fluorophenyl)-5-[4-(methylsulfonyl)phenyl]-2-phenyl-pyridine-3-carbonitrile (B-126);
4-[2-(4-methylpyridin-2-yl)-4-(trifluoromethyl)-1H-imidazol-1-yl]benzenesulfonamide (B-127);
4-[2-(5-methylpyridin-3-yl)-4-(trifluoromethyl)-1H-imidazol-1-yl]benzenesulfonamide (B-128);
4-[2-(2-methylpyridin-3-yl)-4-(trifluoromethyl)-1H-imidazol-1-yl]benzenesulfonamide (B-129);
3-[1-[4-(methylsulfonyl)phenyl]-4-(trifluoromethyl)-1H-imidazol-2-yl]pyridine (B-130);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
4-[2-(6-methylpyridin-3-yl)-4-(trifluoromethyl)-1H-imidazol-1-yl]benzenesulfonamide (B-134);
2-(3,4-difluorophenyl)-1-[4-(methylsulfonyl)phenyl]-4-(trifluoromethyl)-1H-imidazole (B-135);
4-[2-(4-methylphenyl)-4-(trifluoromethyl)-1H-imidazol-1-yl]benzenesulfonamide (B-136);
2-(4-chlorophenyl)-1-[4-(methylsulfonyl)phenyl]-4-methyl-1H-imidazole (B-137);
2-(4-chlorophenyl)-1-[4-(methylsulfonyl)phenyl]-4-phenyl-1H-imidazole (B-138);
2-(4-chlorophenyl)-4-(4-fluorophenyl)-1-[4-(methylsulfonyl)phenyl]-1H-imidazole (B-139);
2-(3-fluoro-4-methoxyphenyl)-1-[4-(methylsulfonyl)phenyl-4-(trifluoro methyl)-1H-imidazole (B-140);
1-[4-(methylsulfonyl)phenyl]-2-phenyl-4-trifluoromethyl-1H-imidazole (B-141);
2-(4-methylphenyl)-1-[4-(methylsulfonyl)phenyl]-4-trifluoromethyl-1H-imidazole (B-142);
4-[2-(3-chloro-4-methylphenyl)-4-(trifluoromethyl)-1H-imidazol-1-yl]benzenesulfonamide (B-143);
2-(3-fluoro-5-methylphenyl)-1-[4-(methylsulfonyl)phenyl]-4-(trifluoro methyl)-1H-imidazole (B-144);
4-[2-(3-fluoro-5-methylphenyl)-4-(trifluoromethyl)-1H-imidazol-1-yl]benzenesulfonamide (B-145);
2-(3-methylphenyl)-1-[4-(methylsulfonyl)phenyl]-4-trifluoromethyl-1H-imidazole (B-146);
4-[2-(3-methylphenyl)-4-trifluoromethyl-1H-imidazol-1-yl]benzene sulfonamide (B-147);
1-[4-(methylsulfonyl)phenyl]-2-(3-chlorophenyl)-4-trifluoromethyl-1H-imidazole (B-148);
4-[2-(3-chlorophenyl)-4-trifluoromethyl-1H-imidazol-1-yl]benzenesulfonamide (B-149);
4-[2-phenyl-4-trifluoromethyl-1H-imidazol-1-yl]benzenesulfonamide (B-150);
4-[2-(4-methoxy-3-chlorophenyl)-4-trifluoromethyl-1H-imidazol-1-yl]benzenesulfonamide (B-151);
1-allyl-4-(4-fluorophenyl)-3-[4-(methylsulfonyl)phenyl]-5-(trifluoro methyl)-1H-pyrazole (B-152);
4-[1-ethyl-4-(4-fluorophenyl)-5-(trifluoromethyl)-1H-pyrazol-3-yl]benzenesulfonamide (B-153);
N-phenyl-[4-(4-fluorophenyl)-3-[4-(methylsulfonyl)phenyl]-5-(trifluoromethyl)-1H-pyrazol-1-yl]acetamide (B-154);
ethyl[4-(4-fluorophenyl)-3-[4-(methylsulfonyl)phenyl]-5-(trifluoromethyl)-1H-pyrazol-1-yl]acetate (B-155);
4-(4-fluorophenyl)-3-[4-(methylsulfonyl)phenyl]-1-(2-phenylethyl)-1H-pyrazole (B-156);
4-(4-fluorophenyl)-3-[4-(methylsulfonyl)phenyl]-1-(2-phenylethyl)-5-(trifluoromethyl)pyrazole (B-157);
1-ethyl-4-(4-fluorophenyl)-3-[4-(methylsulfonyl)phenyl]-5-(trifluoromethylyl)-1H-pyrazole (B-158);
5-(4-fluorophenyl)-4-(4-methylsulfonylphenyl)-2-trifluoromethyl-1H-imidazole (B-159);
4-[4-(methylsulfonyl)phenyl]-5-(2-thiophenyl)-2-(trifluoromethyl)-1H-imidazole (B-160);
5-(4-fluorophenyl)-2-methoxy-4-[4-(methylsulfonyl)phenyl]-6-(trifluoromethyl)pyridine (B-161);
2-ethoxy-5-(4-fluorophenyl)-4-[4-(methylsulfonyl)phenyl]-6-(trifluoromethyl)pyridine (B-162);
5-(4-fluorophenyl)-4-[4-(methylsulfonyl)phenyl]-2-(2-propynyloxy)-6-(trifluoromethyl)pyridine (B-163);
2-bromo-5-(4-fluorophenyl)-4-[4-(methylsulfonyl)phenyl]-6-(trifluoromethyl)pyridine (B-164);
4-[2-(3-chloro-4-methoxyphenyl)-4,5-difluorophenyl]benzenesulfonamide (B-165);
1-(4-fluorophenyl)-2-[4-(methylsulfonyl)phenyl]benzene (B-166);
5-difluoromethyl-4-(4-methylsulfonylphenyl)-3-phenylisoxazole (B-167);
4-[3-ethyl-5-phenylisoxazol-4-yl]benzenesulfonamide (B-168);
4-[5-difluoromethyl-3-phenylisoxazol-4-yl]benzenesulfonamide (B-169);
4-[5-hydroxymethyl-3-phenylisoxazol-4-yl]benzenesulfonamide (B-170);
4-[5-methyl-3-phenyl-isoxazol-4-yl]benzenesulfonamide (B-171);
1-[2-(4-fluorophenyl)cyclopenten-1-yl]-4-(methylsulfonyl)benzene (B-172);
1-[2-(4-fluoro-2-methylphenyl)cyclopenten-1-yl]-4-(methylsulfonyl)benzene (B-173);
1-[2-(4-chlorophenyl)cyclopenten-1-yl]-4-(methylsulfonyl)benzene (B-174);
1-[2-(2,4-dichlorophenyl)cyclopenten-1-yl]-4-(methylsulfonyl)benzene (B-175);
1-[2-(4-trifluoromethylphenyl)cyclopenten-1-yl]-4-(methylsulfonyl)benzene (B-176);
1-[2-(4-methylthiophenyl)cyclopenten-1-yl]-4-(methylsulfonyl)benzene (B-177);
1-[2-(4-fluorophenyl)-4,4-dimethylcyclopenten-1-yl]-4-(methylsulfonyl)benzene (B-178);
4-[2-(4-fluorophenyl)-4,4-dimethylcyclopenten-1-yl]benzene sulfonamide (B-179);
1-[2-(4-chlorophenyl)-4,4-dimethylcyclopenten-1-yl]-4-(methylsulfonyl)benzene (B-180);
4-[2-(4-chlorophenyl)-4,4-dimethylcyclopenten-1-yl]benzene sulfonamide (B-181);
4-[2-(4-fluorophenyl)cyclopenten-1-yl]benzenesulfonamide (B-182);
4-[2-(4-chlorophenyl)cyclopenten-1-yl]benzenesulfonamide (B-183);
1-[2-(4-methoxyphenyl)cyclopenten-1-yl]-4-(methylsulfonyl)benzene (B-184);
1-[2-(2,3-difluorophenyl)cyclopenten-1-yl]-4-(methylsulfonyl)benzene (B-185);
4-[2-(3-fluoro-4-methoxyphenyl)cyclopenten-1-yl]benzenesulfonamide (B-186);
1-[2-(3-chloro-4-methoxyphenyl)cyclopenten-1-yl]-4-(methylsulfonyl)benzene (B-187);
4-[2-(3-chloro-4-fluorophenyl)cyclopenten-1-yl]benzenesulfonamide (B-188);
4-[2-(2-methylpyridin-5-yl)cyclopenten-1-yl]benzenesulfonamide (B-189);
ethyl 2-[4-(4-fluorophenyl)-5-[4-(methylsulfonyl)phenyl]oxazol-2-yl]-2-benzyl-acetate (B-190);
2-[4-(4-fluorophenyl)-5-[4-(methylsulfonyl)phenyl]oxazol-2-yl]acetic acid (B-191);
2-(tert-butyl)-4-(4-fluorophenyl)-5-[4-(methylsulfonyl)phenyl]oxazole (B-192);
4-(4-fluorophenyl)-5-[4-(methylsulfonyl)phenyl]-2-phenyloxazole (B-193);
4-(4-fluorophenyl)-2-methyl-5-[4-(methylsulfonyl)phenyl]oxazole (B-194);
4-[5-(3-fluoro-4-methoxyphenyl)-2-trifluoromethyl-4-oxazolyl]benzenesulfonamide (B-195);
6-chloro-7-(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H)-furanone (B-198);
6-chloro-2-trifluoromethyl-2H-1-benzothiopyran-3-carboxylic acid (B-199);
4-[5-(4-chlorophenyl)-3-(trifluoromethyl)-1H-pyrazol-1-yl]benzene sulfonamide (B-200);
4-[5-(4-methylphenyl)-3-(trifluoromethyl)-1H-pyrazol-1-yl]benzene sulfonamide (B-201);
4-[5-(3-fluoro-4-methoxyphenyl)-3-(difluoromethyl)-1H-pyrazol-1-yl]benzenesulfonamide (B-202);
3-[1-[4-(methylsulfonyl)phenyl]-4-trifluoromethyl-1H-imidazol-2-yl]pyridine (B-203);
2-methyl-5-[1-[4-(methylsulfonyl)phenyl]-4-trifluoromethyl-1H-imidazol-2-yl]pyridine (B-204);
4-[2-(5-methylpyridin-3-yl)-4-(trifluoromethyl)-1H-imidazol-1-yl]benzenesulfonamide (B-205);
4-[5-methyl-3-phenylisoxazol-4-yl]benzenesulfonamide (B-206);
4-[5-hydroxymethyl-3-phenylisoxazol-4-yl]benzenesulfonamide (B-207);
[2-trifluoromethyl-5-(3,4-difluorophenyl)-4-oxazolyl]benzenesulfonamide (B-208);
4-[2-methyl-4-phenyl-5-oxazolyl]benzenesulfonamide (B-209);
4-[5-(2-fluoro-4-methoxyphenyl)-2-trifluoromethyl-4-oxazolyl]benzenesulfonamide (B-210);
[2-(2-chloro-6-fluoro-phenylamino)-5-methyl-phenyl]-acetic acid or COX 189 (lumiracoxib; B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, sodium salt (B-214);
N-[5-(4-fluoro-phenylsulfanyl)-thiophen-2-yl]-methanesulfonamide (B-215);
3-(3,4-Difluoro-phenoxy)-4-(4-methanesulfonyl-phenyl)-5-methyl-5-(2,2,2-trifluoro-ethyl)-5H-furan-2-one (B-216);
(5Z)-2-amino-5-[[3,5-bis(1,1-dimethylethyl)-4-hydroxyphenyl]methylene]-4(5H)-thiazolone (B-217);
CS-502 (B-218);
LAS-34475 (B-219);
LAS-34555 (B-220);
S-33516 (B-221);
SD-8381 (B-222);
L-783003 (B-223);
N-[3-(formylamino)-4-oxo-6-phenoxy-4H-1-benzopyran-7-yl]-methanesulfonamide (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid (B-227);
CGP-28238 (B-228);
4-[[3,5-bis(1,1-dimethylethyl)-4-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231);
S-2474 (B-232);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methyl sulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3
EXAMPLES OF CYCLOOXYGENASE-2 SELECTIVE
INHIBITORS AS EMBODIMENTS

| Com- | |
|---|---|
| pound | |
| Number | Structural Formula |
| B-26 | 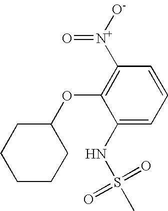 |
| | N-(2-cyclohexyloxynitrophenyl)methane |
| | sulfonamide or NS-398; |
| B-27 | 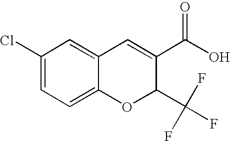 |
| | 6-chloro-2-trifluoromethyl-2H-1-benzopyran-3- |
| | carboxylic acid; |
| B-28 | 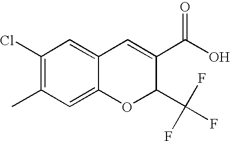 |
| | 6-chloro-7-methyl-2-trifluoromethyl-2H-1- |
| | benzopyran-3-carboxylic acid |
| B-29 | 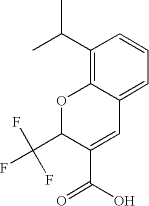 |
| | 8-(1-methylethyl)-2-trifluoromethyl-2H-1- |
| | benzopyran-3-carboxylic acid; |
| B-30 | 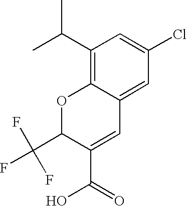 |
| | 6-chloro-8-(1-methylethyl)-2-trifluoromethyl- |
| | 2H-1-benzopyran-3-carboxylic acid; |
| B-31 | 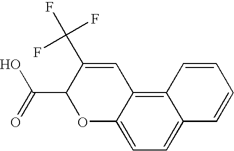 |
| | 2-trifluoromethyl-3H-naphtho[2,1-b]pyran- |
| | 3-carboxylic acid; |
| B-32 | 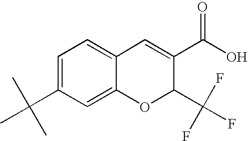 |
| | 7-(1,1-dimethylethyl)-2-trifluoromethyl-2H-1- |
| | benzopyran-3-carboxylic acid; |
| B-33 | 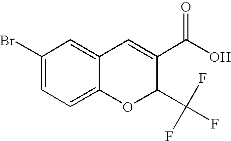 |
| | 6-bromo-2-trifluoromethyl-2H-1-benzopyran- |
| | 3-carboxylic acid; |
| B-34 | 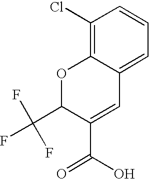 |
| | 8-chloro-2-trifluoromethyl-2H-1-benzopyran- |
| | 3-carboxylic acid; |
| B-35 | 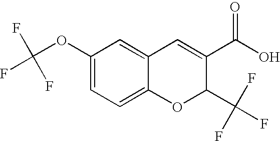 |
| | 6-trifluoromethoxy-2-trifluoromethyl-2H-1- |
| | benzopyran-3-carboxylic acid; |
| B-36 | 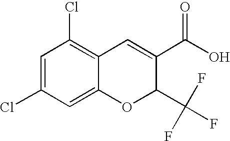 |
| | 5,7-dichloro-2-trifluoromethyl-2H-1- |
| | benzopyran-3-carboxylic acid; |
| B-37 | 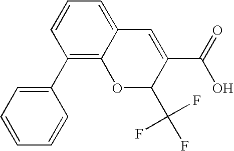 |
| | 8-phenyl-2-trifluoromethyl-2H-1-benzopyran- |
| | 3-carboxylic acid; |
| B-38 | 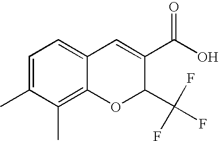 |
| | 7,8-dimethyl-2-trifluoromethyl-2H-1-benzopyran- |
| | 3-carboxylic acid; |
| B-39 | 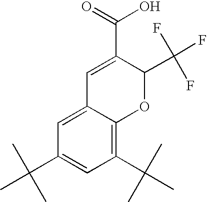 |
| | 6,8-bis(dimethylethyl)-2-trifluoromethyl-2H-1- |
| | benzopyran-3-carboxylic acid; |
| B-40 | 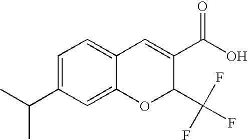 |
| | 7-(1-methylethyl)-2-trifluoromethyl-2H-1- |
| | benzopyran-3-carboxylic acid; |
| B-41 | 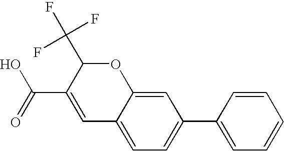 |
| | 7-phenyl-2-trifluoromethyl-2H-1-benzopyran- |
| | 3-carboxylic acid; |
| B-42 | 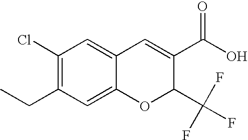 |
| | 6-chloro-7-ethyl-2-tritluoromethyl-2H-1- |
| | benzopyran-3-carboxylic acid; |
| B-43 | 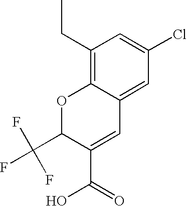 |
| | 6-chloro-8-ethyl-2-trfluoromethyl-2H-1- |
| | benzopyran-3-carboxylic acid; |
| B-44 | 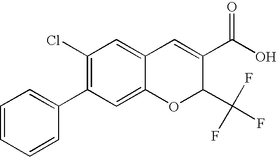 |
| | 6-chloro-7-phenyl-2-trifluoromethyl-2H-1- |
| | benzopyran-3-carboxylic acid; |
| B-45 | 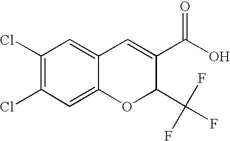 |
| | 6,7-dichloro-2-trifluoromethyl-2H-1- |
| | benzopyran-3-carboxylic acid; |
| B-46 | 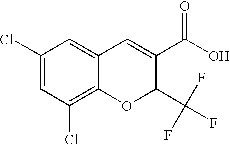 |
| | 6,8-dichloro-2-trifluoromethyl-2H-1- |
| | benzopyran-3-carboxylic acid; |
| B-47 | 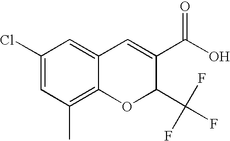 |
| | 6-chloro-8-methyl-2-trifluoromethyl-2H-1- |
| | benzopyran-3-carboxylic acid; |
| B-48 | 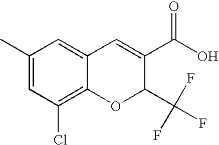 |
| | 8-chloro-6-methyl-2-trifluoromethyl-2H-1- |
| | benzopyran-3-carboxylic acid; |
| B-49 | 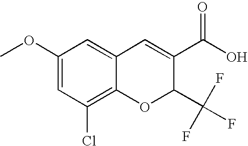 |
| | 8-chloro-6-methoxy-2-trifluoromethyl-2H-1- |
| | benzopyran-3-carboxylic acid; |
| B-50 | 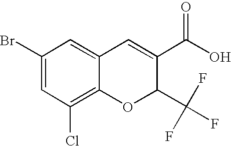 |
| | 6-bromo-8-chloro-2-trifluoromethyl-2H-1- |
| | benzopyran-3-carboxylic acid; |
| B-51 | 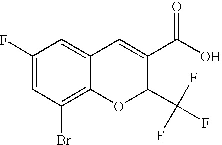 |
| | 8-bromo-6-fluoro-2-trifluoromethyl-2H-1- |
| | benzopyran-3-carboxylic acid; |
| B-52 | 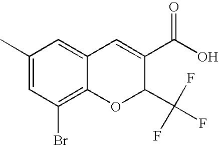 |
| | 8-bromo-6-methyl-2-trifluoramethyl-2H-1- |
| | benzopyran-3-carboxylic acid; |
| B-53 | 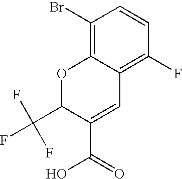 |
| | 8-bromo-5-fluoro-2-trifluoromethyl-2H-1- |
| | benzopyran-3-carboxylic acid; |
| B-54 | 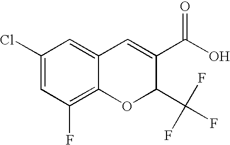 |
| | 6-chloro-8-fluoro-2-trifluoromethyl-2H-1- |
| | benzopyran-3-carboxylic acid; |
| B-55 | 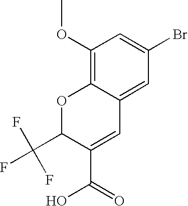 |
| | 6-bromo-8-methoxy-2-trifluoromethyl-2H-1- |
| | benzopyran-3-carboxylic acid; |
| B-56 | 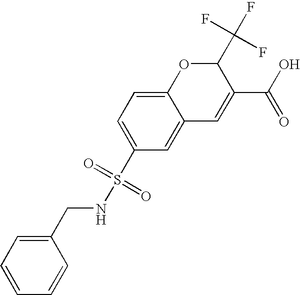 |
| | 6-[[(phenylmethyl)amino]sulfonyl]-2- |
| | trifluoromethyl-2H-1-benzopyran-3-carboxylic acid; |
| B-57 | 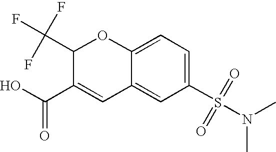 |
| | 6-[(dimethylamino)sulfonyl]-2-trifluoromethyl- |
| | 2H-1-benzopyran-3-carboxylic acid; |
| B-58 | 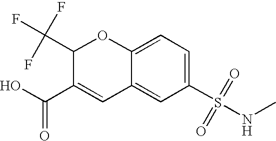 |
| | 6-[(methylamino)sulfonyl]-2-trifluoromethyl-2H- |
| | 1-benzopyran-3-carboxylic acid; |
| B-59 | 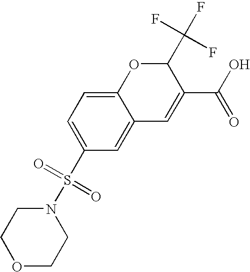 |
| | 6-[(4-morpholino)sulfonyl]-2-trifluoromethyl- |
| | 2H-1-benzopyran-3-carboxylic acid; |
| B-60 | 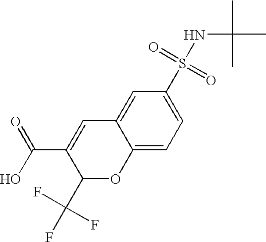 |
| | 6-[(1,1-dimethylethyl)aminosulfonyl]-2-trifluoromethyl- |
| | 2H-1-benzopyran-3-carboxylic acid; |
| B-61 | 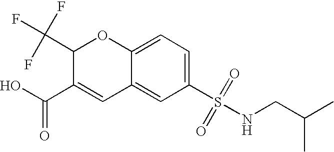 |
| | 6-[(2-methylpropyl)aminosulfonyl]-2-trifluoromethyl- |
| | 2H-1-benzopyran-3-carboxylic acid; |
| B-62 | 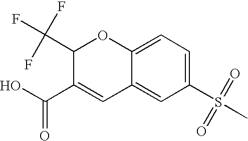 |
| | 6-methylsulfonyl-2-trifluoromethyl-2H-1-benzopyran-3- |
| | carboxylic acid; |
| B-63 | 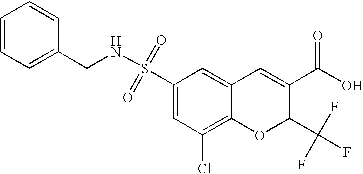 |
| | 8-chloro-6-[[(phenylmethyl)amino]sulfonyl]-2- |
| | trifluoromethyl-2H-1-benzopyran-3-carboxylic acid; |
| B-64 | 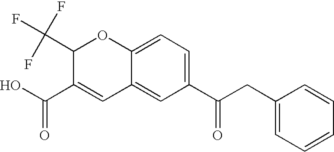 |
| | 6-phenylacetyl-2-trifluoromethyl-2H-1-benzopyran-3- |
| | carboxylic acid; |
| B-65 | 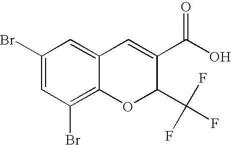 |
| | 6,8-dibromo-2-trifluoromethyl-2H-1-benzopyran-3- |
| | carboxylic acid; |
| B-66 | 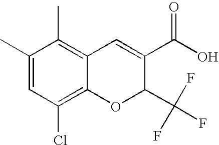 |
| | 8-chloro-5,6-dimethyl-2-trifluoromethyl-2H-1- |
| | benzopyran-3-carboxylic acid; |
| B-67 | 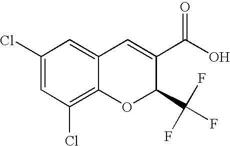 |
| | 6,8-dichloro-(S)-2-trifluoromethyl-2H-1-benzopyran-3- |
| | carboxylic acid; |
| B-68 | 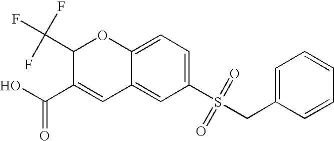 |
| | 6-benzylsulfonyl-2-trifluoromethyl-2H-1-benzopyran-3- |
| | carboxylic acid; |
| B-69 | 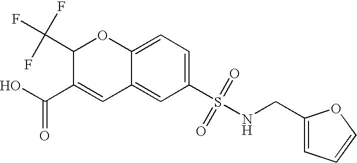 |
| | 6-[[N-(2-furylmethyl)amino]sulfonyl]-2-trifluoromethyl- |
| | 2H-1-benzopyran-3-carboxylic acid; |
| B-70 | 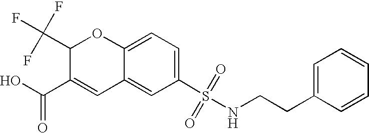 |
| | 6-[[N-(2-phenylethyl)amino]sulfonyl]-2-trifluoromethyl- |
| | 2H-1-benzopyran-3-carboxylic acid |
| B-71 | 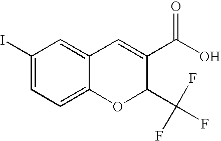 |
| | 6-iodo-2-trifluoromethyl-2H-1-benzopyran-3- |
| | carboxylic acid; |
| B-72 | 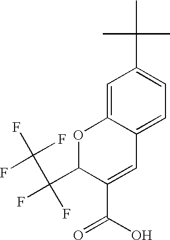 |
| | 7-(1,1-dimethylethyl)-2-pentafluoroethyl-2H- |
| | 1-benzopyran-3-carboxylic acid; |
| B-73 | 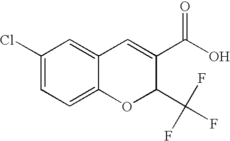 |
| | 6-chloro-2-trifluoromethyl-2H-1-benzothiopyran-3- |
| | caxboxylic acid; |
| B-74 | 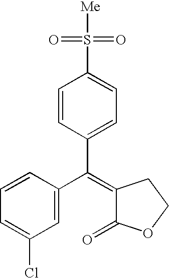 |
| | 3-[(3-chloro-phenyl)-(4-methanesulfonyl-phenyl)-methylene]- |
| | dihydro-furan-2-one or BMS-347070; |
| B-75 | 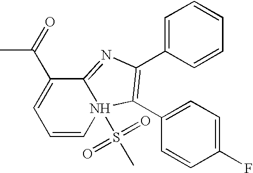 |
| | 8-acetyl-3-(4-fluorophenyl)-2-(4-methylsulfonyl)phenyl- |
| | imidazo(1,2-a)pyridine; |
| B-76 | 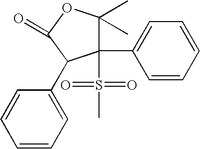 |
| | 5,5-dimethyl-4-(4-methylsulfonyl)phenyl-3-phenyl-2-(5H)- |
| | furanone; |
| B-77 | 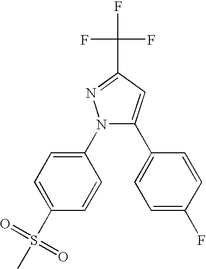 |
| | 5-(4-fluorophenyl)-1-[4-(methylsulfonyl)phenyl]-3- |
| | (trifluoromethyl)pyrazole; |
| B-78 | 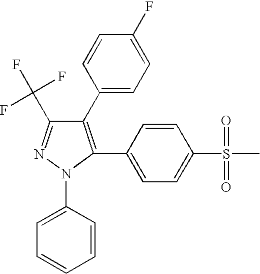 |
| | 4-(4-fluorophenyl)-5-[4-(methylsulfonyl)phenyl]- |
| | 1-phenyl-3-(trifluoromethyl)pyrazole; |
| B-79 | 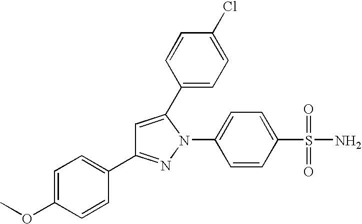 |
| | 4-(5-(4-chlorophenyl)-3-(4-methoxyphenyl)-1H-pyrazol-1-yl) |
| | benzenesulfonamide; |
| B-80 | 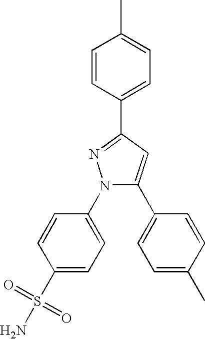 |
| | 4-(3,5-bis(4-methylphenyl)-1H-pyrazol-1- |
| | yl)benzenesulfonamide; |
| B-81 | 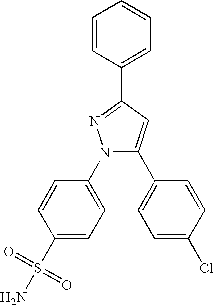 |
| | 4-(5-(4-chlorophenyl)-3-phenyl-1H-pyrazol-1- |
| | yl)benzenesulfonamide; |
| B-82 | 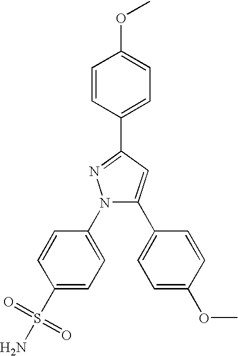 |
| | 4-(3,5-bis(4-methoxyphenyl)-1H-pyrazol-1- |
| | yl)benzenesulfonamide; |
| B-83 | 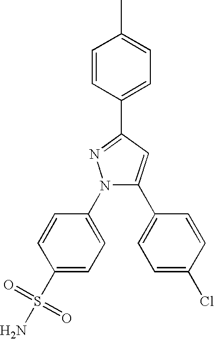 |
| | 4-(5-(4-chlorophenyl)-3-(4-methylphenyl)-1H- |
| | pyrazol-1-yl)benzenesulfonamide; |
| B-84 | 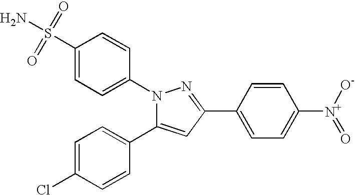 |
| | 4-(5-(4-chlorophenyl)-3-(4-nitrophenyl)-1H- |
| | pyrazol-1-yl)benzenesulfonamide; |
| B-85 | 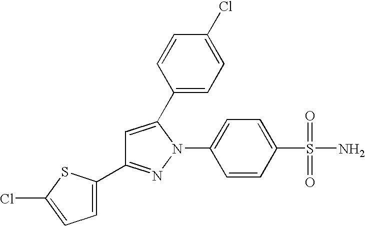 |
| | 4-(5-(4-chlorophenyl)-3-(5-chloro-2-thienyl)-1H- |
| | pyrazol-1-y)benzenesulfonamide; |
| B-86 | 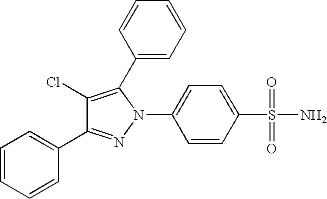 |
| | 4-(4-chloro-3,5-diphenyl-1H-pyrazol-1- |
| | yl)benzenesulfonamide; |
| B-87 | 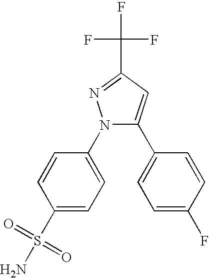 |
| | 4-[5-(4-chlorophenyl)-3-(trifluoromethyl)-1H- |
| | pyrazol-1-yl]benzenesulfonamide; |
| B-88 | 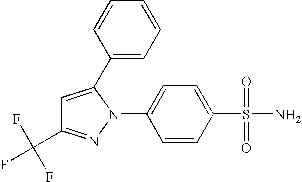 |
| | 4-[5-phenyl-3-(trifluoromethyl)-1H-pyrazol-1- |
| | yl]benzenesulfonamide; |
| B-89 | 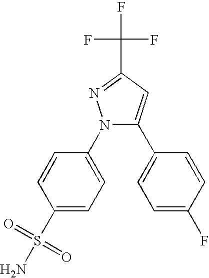 |
| | 4-[5-(4-fluorophenyl)-3-(trifluoromethyl)-1H- |
| | pyrazol-1-yl]benzenesulfonamide; |
| B-90 | 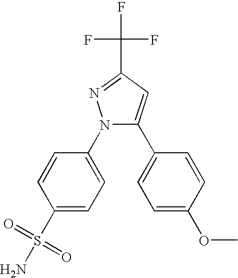 |
| | 4[5-(4-methoxyphenyl)-3-(trifluoromethyl)-1H- |
| | pyrazol-1-yl]benzenesulfonamide; |
| B-91 | 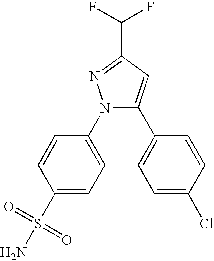 |
| | 4-[5-(4-chlorophenyl)-3-(difluoromethyl)-1H- |
| | pyrazol-1-yl]benzenesulfonamide; |
| B-92 | 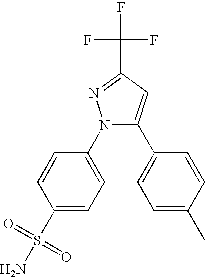 |
| | 4-[5-(4-methylphenyl)-3-(trifluoromethyl)-1H- |
| | pyrazol-1-yl]benzenesulfonamide; |
| B-93 | 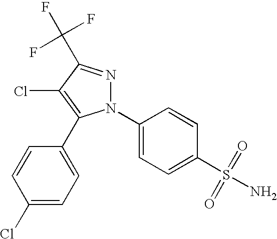 |
| | 4-[4-chloro-5-(4-chlorophenyl)-3-(trifluoromethyl)-1H- |
| | pyrazol-1-yl]benzenesulfonamide; |
| B-94 | 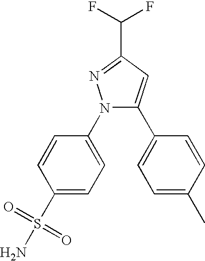 |
| | 4-[3-(difluoromethyl)-5-(4-methylphenyl)-1H-pyrazol-1- |
| | yl]benzenesulfonamide; |
| B-95 | 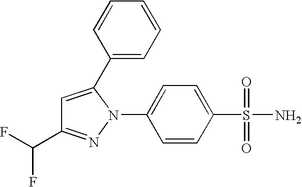 |
| | 4-[3-(difluoromethyl)-5-phenyl-1H-pyrazol-1- |
| | yl]benzenesulfonamide; |
| B-96 | 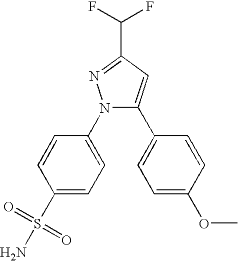 |
| | 4-[3-(difluorometbyl)-5-(4-methoxyphenyl)-1H- |
| | pyrazol-1-yl]benzenesulfonamide; |
| B-97 | 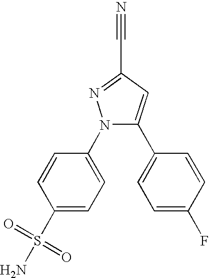 |
| | 4-[3-cyano-5-(4-fluorophenyl)-1H-pyrazol-1- |
| | yl]benzenesulfonamide; |
| B-98 | 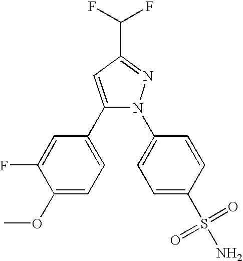 |
| | 4-[3-(difluoromethyl)-5-(3-fluoro-4-methoxyphenyl)-1H- |
| | pyrazol-1-yl]benzenesulfonamide; |
| B-99 | 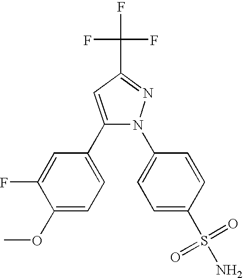 |
| | 4-[5-(3-fluoro-4-methoxyphenyl)-3-(trifluoromethyl)-1H- |
| | pyrazol-1-yl]benzenesulfonamide; |
| B-100 | 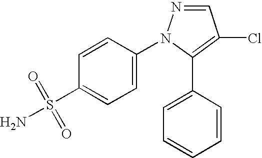 |
| | 4-[4-chloro-5-phenyl-1H-pyrazol-1- |
| | yl]benzenesulfonamide; |
| B-101 | 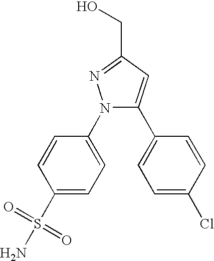 |
| | 4-[5-(4-chlorophenyl)-3-(hydroxymethyl)-1H-pyrazol-1- |
| | yl]benzenesulfonamide; |
| B-102 | 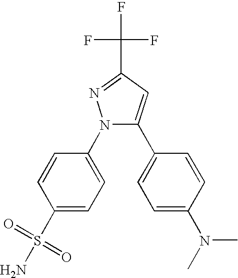 |
| | 4-[5-(4-(N,N-dimethylamino)phenyl)-3-(trifluoromethyl)- |
| | 1H-pyrazol-1-yl]benzenesulfonamide; |
| B-103 | 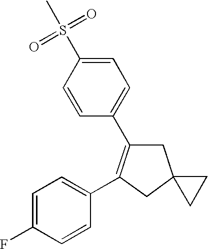 |
| | 5-(4-fluorophenyl)-6-[4-(methylsulfonyl)phenyl] |
| | spiro[2.4]hept-5-ene; |
| B-104 | 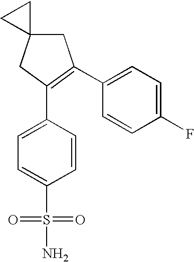 |
| | 4-[6-(4-fluorophenyl)spiro[2.4]hept-5-en-5- |
| | yl]benzenesulfonamide; |
| B-105 | 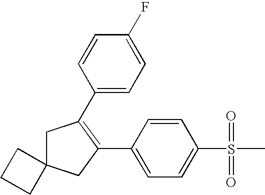 |
| | 6-(4-fluorophenyl)-7-[4-methylsulfonyl) |
| | phenyl]spiro[3.4]oct-6-ene; |
| B-106 | 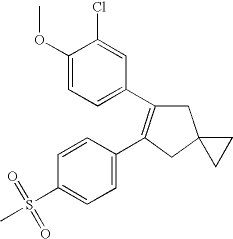 |
| | 5-(3-chloro-4-methoxyphenyl)-6-[4- |
| | (methylsulfonyl)phenyl]spiro[2.4]hept-5-ene; |
| B-107 | 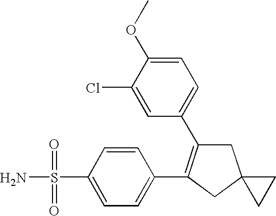 |
| | 4-[6-(3-chloro-4-methoxyphenyl)spiro[2.4]hept-5-en-5- |
| | yl]benzenesulfonamide; |
| B-108 | 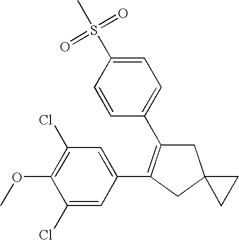 |
| | 5-(3,5-dichloro-4-methoxyphenyl)-6-[4-(methylsulfonyl) |
| | phenyl]spiro[2.4]hept-5-ene; |
| B-109 | 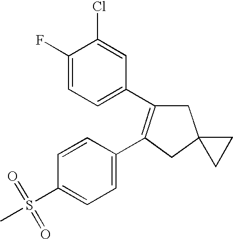 |
| | 5-(3-chloro-4-fluorophenyl)-6-(4-(methylsulfonyl)phenyl] |
| | spiro[2.4]hept-5-ene; |

| Compound | |
|---|---|
| Number | Structural Formula |
| B-110 | 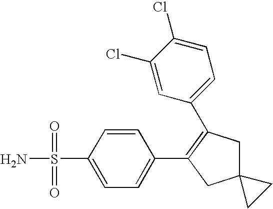 |
| | 4-[6-(3,4-dichlorophenyl)spiro[2.4]hept-5-en-5- |
| | yl]benzenesulfonamide; |
| B-111 | 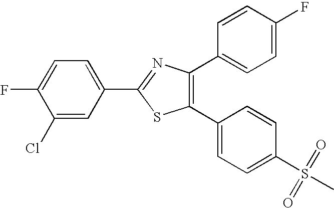 |
| | 2-(3-chloro-4-fluorophenyl)-4-(4-fluorophenyl)-5-(4- |
| | methylsulfonylphenyl)thiazole; |
| B-112 | 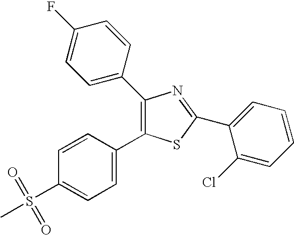 |
| | 2-(2-chlorophenyl)-4-(4-fluorophenyl)-5-(4- |
| | methylsulfonylphenyl)thiazole; |
| B-113 | 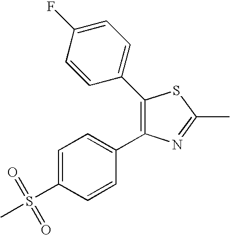 |
| | 5-(4-fluorophenyl)-4-(4-methylsulfonylphenyl)-2- |
| | methylthiazole; |
| B-114 | 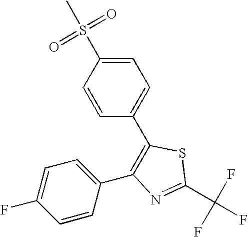 |
| | 4-(4-fluorophenyl)-5-(4-methylsulfonylphenyl)-2- |
| | trifluoromethylthiazole; |
| B-115 | 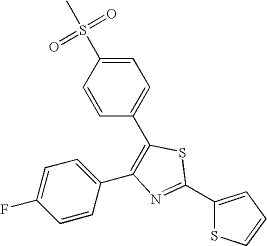 |
| | 4-(4-fluorophenyl)-5-(4-methylsulfonylphenyl)-2-(2- |
| | thienyl)thiazole; |
| B-116 | 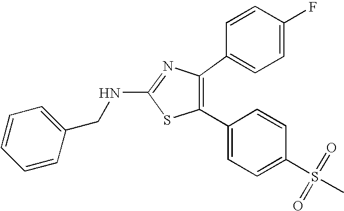 |
| | 4-(4-fluorophenyl)-5-(4-methylsulfonylphenyl)-2- |
| | benzylaminothiazole; |
| B-117 | 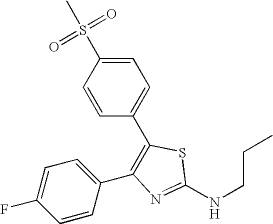 |
| | 4-(4-fluorophenyl)-5-(4-methylsulfonylphenyl)-2-(1- |
| | propylamino)thiazole; |
| B-118 | 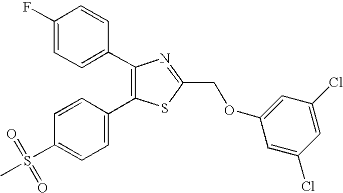 |
| | 2-((3,5-chlorophenoxy)methyl)-4-(4-fluorophenyl)-5-[4- |
| | (methylsulfonyl)phenyl]thiazole; |
| B-119 | 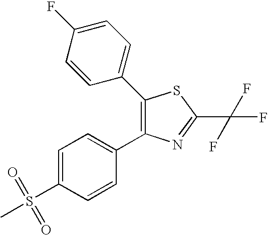 |
| | 5-(4-fluorophenyl)-4-(4-methylsulfonylphenyl)-2- |
| | trifluoromethylthiazole; |
| B-120 | 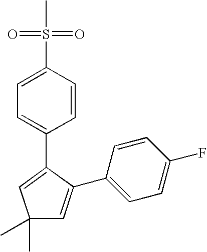 |
| | 1-methylsulfonyl-4-[1,1-dimethyl-4-(4-fluorophenyl) |
| | cyclopenta-2,4-dien-3-yl]benzene; |
| B-121 | 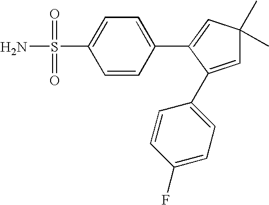 |
| | 4-[4-(4-fluorophenyl)-1,1-dimethylcyclopenta-2,4-dien- |
| | 3-yl]benzenesulfonamide; |
| B-122 | 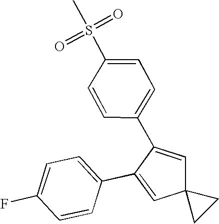 |
| | 5-(4-fluorophenyl)-6-[4-(methylsulfonyl)phenyl] |
| | spiro[2.4]hepta-4,6-diene; |
| B-123 | 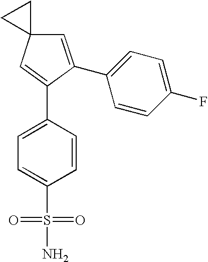 |
| | 4-[6-(4-fluorophenyl)spiro[2.4]hepta-4,6-dien-5- |
| | yl]benzenesulfonamide; |
| B-124 | 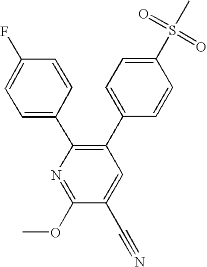 |
| | 6-(4-fluorophenyl)-2-methoxy-5-[4-(methylsulfonyl) |
| | phenyl]-pyridine-3-carbonitrile; |
| B-125 | 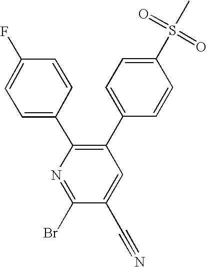 |
| | 2-bromo-6-(4-fluorophenyl)-5-[4-(methylsulfonyl)phenyl]- |
| | pyridine-3-carbonitrile; |
| B-126 | 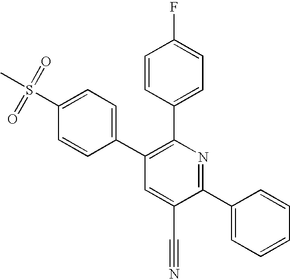 |
| | 6-(4-fluorophenyl)-5-[4-(methylsulfonyl)phenyl]-2-phenyl- |
| | pyridine-3-carbonitrile; |
| B-127 | 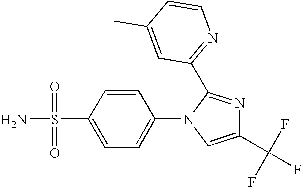 |
| | 4-[2-(4-methylpyridin-2-yl)-4-(trifluoromethyl)-1H- |
| | imidazol-1-yl]benzenesulfonamide; |
| B-128 | 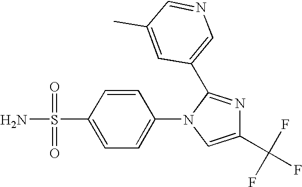 |
| | 4-[2-(5-methylpyridin-3-yl)-4-(trifluoromethyl)-1H- |
| | imidazol-1-yl]benzenesulfonamide; |
| B-129 | 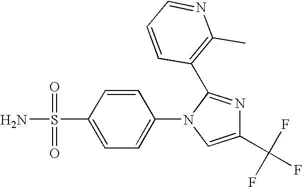 |
| | 4-[2-(2-methylpyridin-3-yl)-4-(trifluoromethyl)-1H- |
| | imidazol-1 -yl]benzenesulfonamide; |
| B-130 | 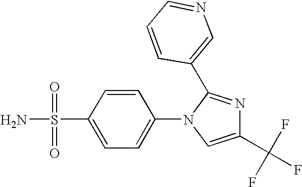 |
| | 3-[1-[4-(methylsulfonyl)phenyl]-4-(trifluoromethyl)- |
| | 1H-imidazol-2-yl]pyridine; |
| B-131 | 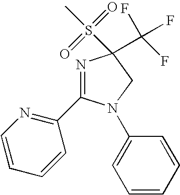 |
| | 2-[1-[4-(methylsulfonyl)phenyl-4-(trifluoromethyl)]-1H- |
| | imidazol-2-yl]pyridine; |
| B-132 | 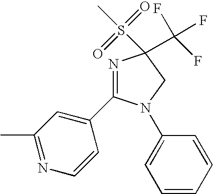 |
| | 2-methyl-4-[1-[4-(methylsulfonyl)phenyl-4- |
| | (trifluoromethyl)]-1H-imidazol-2-yl]pyridine; |
| B-133 | 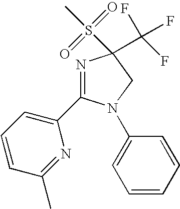 |
| | 2-methyl-6-[1-[4-(methylsulfonyl)phenyl-4- |
| | (trifluoromethyl)]-1H-imidazol-2-yl]pyridine; |
| B-134 | 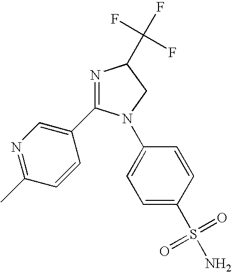 |
| | 4-[2-(6-methylpyridin-3-yl)-4-(trifluoromethyl)-1H- |
| | imidazol-1-yl]benzenesulfonamide; |
| B-135 | 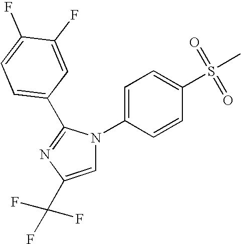 |
| | 2-(3,4-difluorophenyl)-1-[4-(methylsulfonyl)phenyl]- |
| | 4-(trifluoromethyl)-1H-imidazole; |
| B-136 | 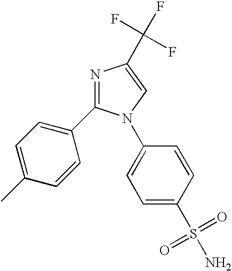 |
| | 4-[2-(4-methylphenyl)-4-(trifluoromethyl)-1H-imidazol-1- |
| | yl]benzenesulfonamide; |
| B-137 | 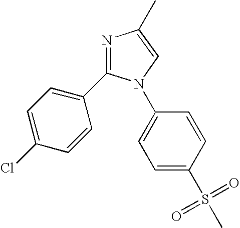 |
| | 2-(4-chlorophenyl)-1-[4-(methylsulfonyl)phenyl]-4- |
| | methyl-1H-imidazole; |
| B-138 | 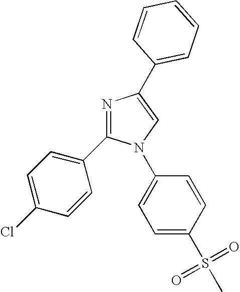 |
| | 2-(4-chlorophenyl)-1-[4-(methylsulfonyl)phenyl]-4- |
| | phenyl-1H-imidazole; |
| B-139 | 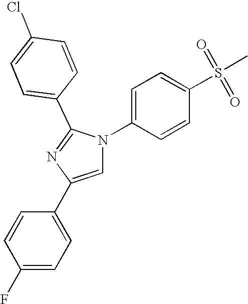 |
| | 2-(4-chlorophenyl)-4-(4-fluorophenyl)-1-[4- |
| | (methylsulfonyl)phenyl]-1H-imidazole; |
| B-140 | 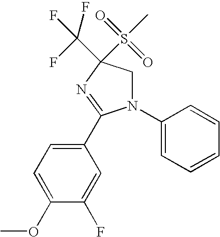 |
| | 2-(3-fluoro-4-methoxyphenyl)-1-[4- |
| | (methylsulfonyl)phenyl-4-(trifluoromethyl)]-1H-imidazole; |
| B-141 | 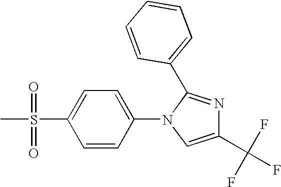 |
| | 1-[4-(methylsulfonyl)phenyl]-2-phenyl-4- |
| | trifluoromethyl-1H-imidazole; |
| B-142 | 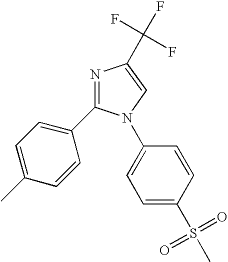 |
| | 2-(4-methylphenyl)-1-[4-(metbylsulfonyl)phenyl]-4- |
| | trifluoromethyl-1H-imidazole; |
| B-143 | 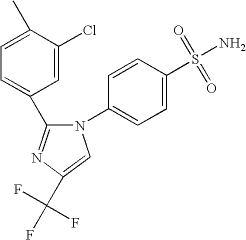 |
| | 4-[2-(3-chloro-4-methylphenyl)-4-(trifluoromethyl)- |
| | 1H-imidazol-1-yl]benzenesulfonamide; |
| B-144 | 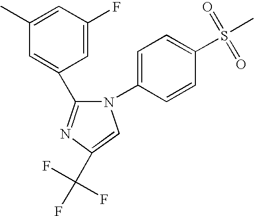 |
| | 2-(3-fluoro-5-methylphenyl)-1-[4-(methylsulfonyl)phenyl]- |
| | 4-(trifluoromethyl)-1H-imidazole; |
| B-145 | 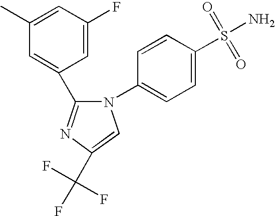 |
| | 4-[2-(3-fluoro-5-methylphenyl)-4-(trifluoromethyl-1H- |
| | imidazole-1-yl]benzenesulfonamide; |
| B-146 | 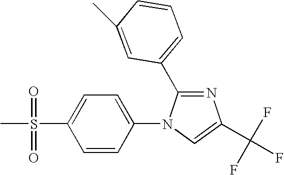 |
| | 2-(3-methylphenyl)-1-[4-(methylsulfonyl)phenyl]-4- |
| | trifluoromethyl-1H-imidazole; |
| B-147 | 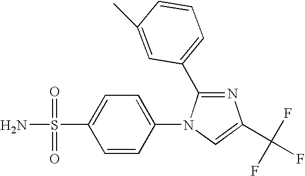 |
| | 4-[2-(3-methylphenyl)-4-trifluoromethyl-1H-imidazol-1- |
| | yl]benzenesulfonamide; |
| B-148 | 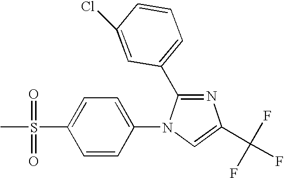 |
| | 1-[4-(methylsulfonyl)phenyl]-2-(3-chlorophenyl)-4- |
| | trifluoromethyl-1H-imidazole |
| B-149 | 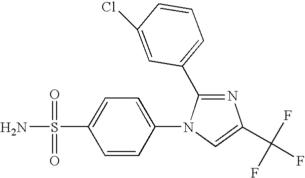 |
| | 4-[2-(3-chlorophenyl)-4-trifluoromethyl-1H-imidazol-1- |
| | yl]benzenesulfonamide; |
| B-150 | 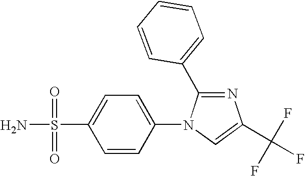 |
| | 4-[2-phenyl-4-trifluoromethyl-1H-imidazol-1- |
| | yl]benzenesulfonamide; |
| B-151 | 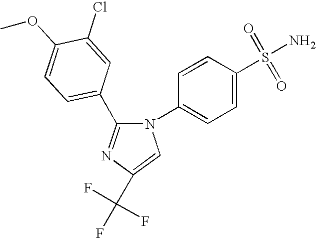 |
| | 4-[2-(4-mexhoxy-3-chlorophenyl)-4-trifluoromethyl-1H- |
| | imidazol-1-yl]benzenesulfonamide; |
| B-152 | 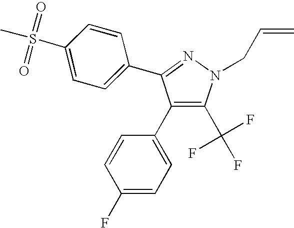 |
| | 1-allyl-4-(4-fluorophenyl)-3-[4-(methylsulfonyl)phenyl]- |
| | 5-(trifluoromethyl)-1H-pyrazole; |
| B-153 | 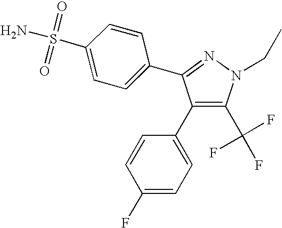 |
| | 4-[1-ethyl-4-(4-fluorophenyl)-5-(trifluoromethyl)-1H- |
| | pyrazol-3-yl]benzenesulfonamide; |
| B-154 | 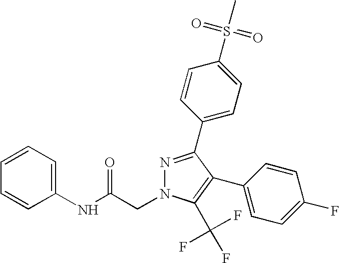 |
| | N-phenyl-[4-(4-fluorophenyl)-3-[4-(methylsulfonyl)phenyl]- |
| | 5-(trifluoromethyl)-1H-pyrazol-1-yl]acetamide; |
| B-155 | 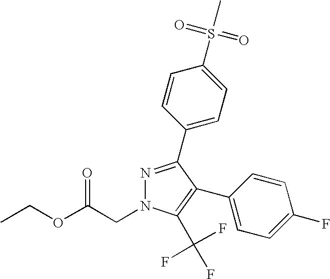 |
| | ethyl[4-(4-fluorophenyl)-3-[4-(methylsulfonyl)phenyl]- |
| | 5-(trifluoromethyl)-1H-pyrazol-1-yl]acetate; |
| B-156 | 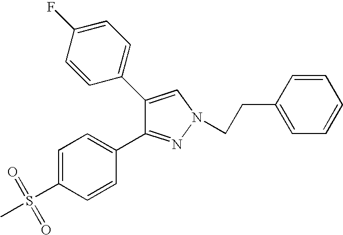 |
| | 4-(4-fluorophenyl)-3-[4-(methylsulfonyl)phenyl]-1-(2- |
| | phenylethyl)-1H-pyrazole; |
| B-157 | 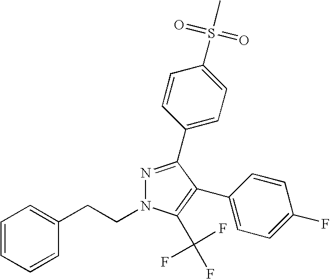 |
| | 4-(4-fluorophenyl)-3-[4-(methylsulfonyl)phenyl]- |
| | 1-(2-phenylethyl)-5-(trifluoromethyl)pyrazole; |
| B-158 | 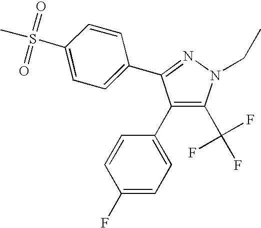 |
| | 1-ethyl-4-(4-fluorophenyl)-3-[4-methylsulfonyl)phenyl]- |
| | 5-(trifluoromethyl)-1H-pyrazole; |
| B-159 | 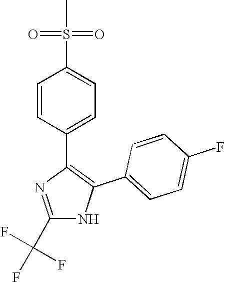 |
| | 5-(4-fluorophenyl)-4-(4-methylsulfonylphenyl)- |
| | 2-trifluoromethyl-1H-imidazole; |
| B-160 | 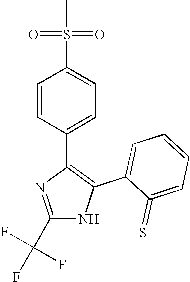 |
| | 4-[4-(methylsulfonyl)phenyl]-5-(2-thiophenyl)-2- |
| | (trifluoromethyl)-1H-imidazole; |
| B-161 | 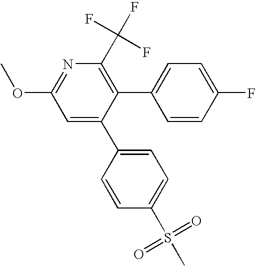 |
| | 5-(4-fluorophenyl)-2-methoxy-4-[4-(methylsulfonyl) |
| | phenyl]-6-(trifluoromethyl)pyridine; |
| B-162 | 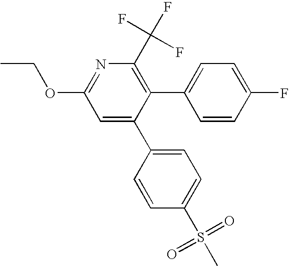 |
| | 2-ethoxy-5-(4-fluorophenyl)-4-[4-(methylsulfonyl)phenyl]- |
| | 6-(trifluoromethyl)pyridine; |
| B-163 | 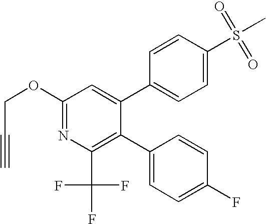 |
| | 5-(4-fluorophenyl)-4-[4-(methylsulfonyl)phenyl]- |
| | 2-(2-propynyloxy)-6-(trifluoromethyl)pyridine; |
| B-164 | 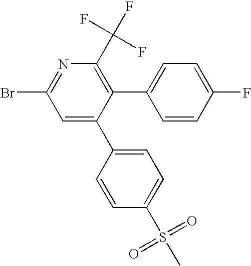 |
| | 2-bromo-5-(4-fluorophenyl)-4-[4-(methylsulfonyl)phenyl]- |
| | 6-(trifluoromethyl)pyridine; |
| B-165 | 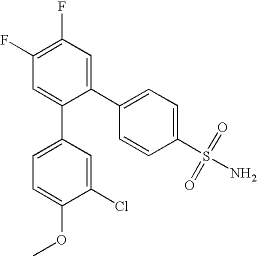 |
| | 4-[2-(3-chloro-4-methoxyphenyl)-4,5-difluorophenyl] |
| | benzenesulfonamide; |
| B-166 | 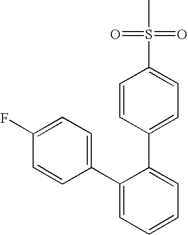 |
| | 1-(4-fluorophenyl)-2-[4-methylsulfonyl)phenyl]benzene; |
| B-167 | 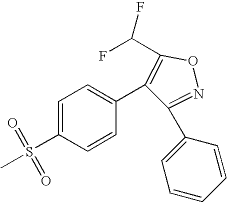 |
| | 5-difluoromethyl-4-(4-methylsulfonylphenyl)-3- |
| | phenylisoxazole; |
| B-168 | 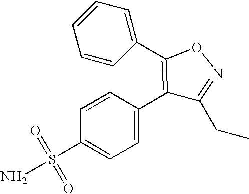 |
| | 4-[3-ethyl-5-phenylisoxazol-4-yl]benzenesulfonamide; |
| B-169 | 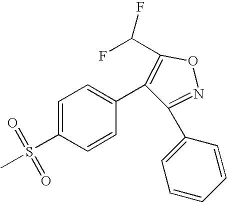 |
| | 4-[5-difluoromethyl-3-phenylisoxazol-4- |
| | yl]benzenesulfonamide; |
| B-170 | 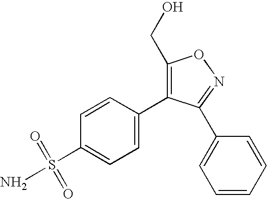 |
| | 4-[5-hydroxymethyl-3-phenylisoxazol-4- |
| | yl]benzenesulfonamide; |
| B-171 | 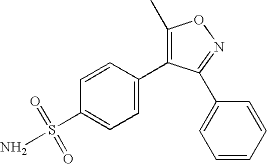 |
| | 4-[5-methyl-3-phenyl-isoxazol-4-yl]benzenesulfonamide; |
| B-172 | 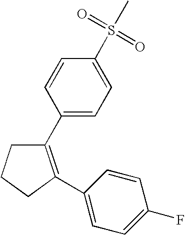 |
| | 1-[2-(4-fluorophenyl)cyclopenten-1-yl]-4- |
| | (methylsulfonyl)benzene; |

| Compound | |
|---|---|
| Number | Structural Formula |
| B-173 | 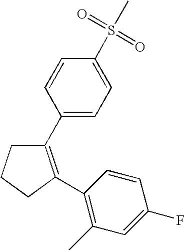 |
| | 1-[2-(4-fluoro-2-methylphenyl)cyclopenten-1- |
| | yl]-4-(methylsulfonyl)benzene; |
| B-174 | 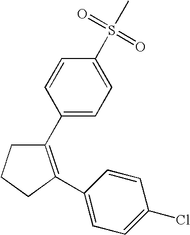 |
| | 1-[2-(4-chlorophenyl)cyclopenten-1-yl]-4- |
| | (methylsulfonyl)benzene; |
| B-175 | 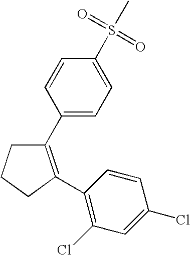 |
| | 1-[2-(2,4-dichlorophenyl)cyclopenten-1-yl]-4- |
| | (methylsulfonyl)benzene; |
| B-176 | 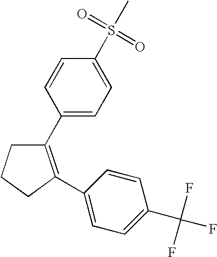 |
| | 1-[2-(4-trifloromethylphenyl)cyclopenten-1-yl]- |
| | 4-(methylsulfonyl)benzene; |
| B-177 | 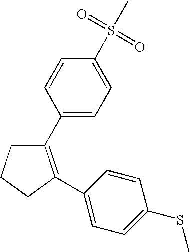 |
| | 1-[2-(4-methylthiophenyl)cyclopenten-1-yl]-4- |
| | (methylsulfonyl)benzene; |
| B-178 | 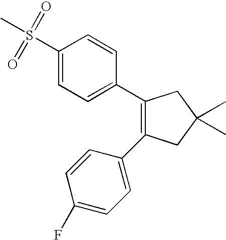 |
| | 1-[2-(4-fluorophenyl)-4,4-dimethylcyclopenten-1- |
| | yl]-4-(methylsulfonyl)benzene; |
| B-179 | 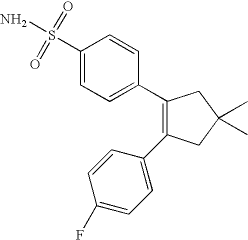 |
| | 4-[2-(4-fluorophenyl)-4,4-dimethylcyclopenten-1- |
| | yl]benzenesulfonamide; |
| B-180 | 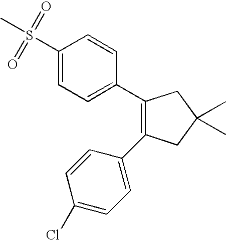 |
| | 1-[2-(3-chlorophenyl)-4,4-dimethylcyclopenten-1- |
| | yl]-4-(methylsulfonyl)benzene; |
| B-181 | 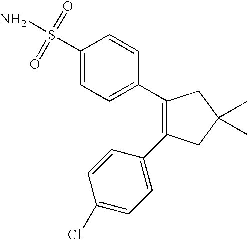 |
| | 4-[2-(4-chlorophenyl)-4,4-dimethylcyclopenten-1- |
| | yl]benzenesulfonamide; |
| B-182 | 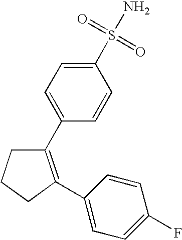 |
| | 4-[2-(4-fluorophenyl)cyclopenten-1- |
| | yl]benzenesulfonamide; |
| B-183 | 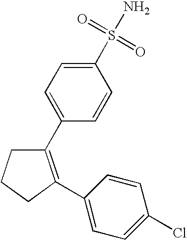 |
| | 4-[2-(4-chlorophenyl)cyclopenten-1- |
| | yl]benzenesulfonamide; |
| B-184 | 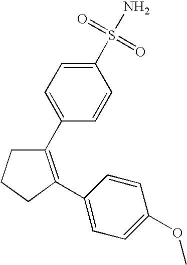 |
| | 1-[2-(4-methoxyphenyl)cyclopenten-1-yl]-4- |
| | (methylsulfonyl)benzene; |
| B-185 | 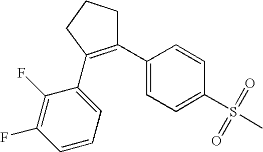 |
| | 1-[2-(2,3-difluorophenyl)cyclopenten-1-yl]-4- |
| | (methylsulfonyl)benzene; |
| B-186 | 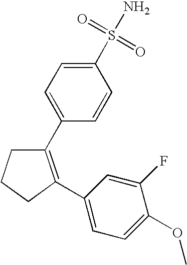 |
| | 4-[2-(3-fluoro-4-methoxyphenyl)cyclopenten-1- |
| | yl]benzenesulfonamide; |
| B-187 | 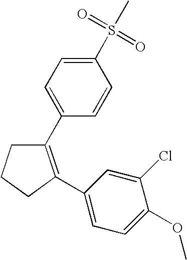 |
| | 1-[2-(3-chloro-4-methoxyphenyl)cyclopenten-1- |
| | yl]-4-(methylsulfonyl)benzene; |
| B-188 | 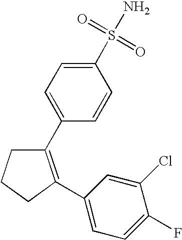 |
| | 4-[2-(3-chloro-4-fluorophenyl)cyclopenten-1- |
| | yl]benzenesulfonamide; |
| B-189 | 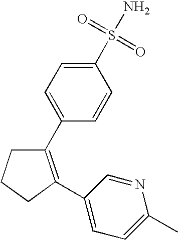 |
| | 4-[2-(2-methylpyridin-5-yl)cyclopenten-1- |
| | yl]benzenesulfonamide; |
| B-190 | 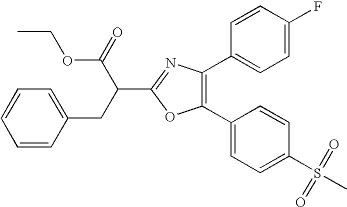 |
| | ethyl 2-[4-(4-fluorophenyl)-5-[4- |
| | (methylsulfonyl)phenyl]oxazol-2-yl]- |
| | 2-benzyl-acetate; |
| B-191 | 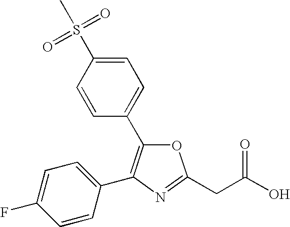 |
| | 2-[4-(4-fluorophenyl)-5-[4-(methyl- |
| | sulfonyl)phenyl]oxazol-2-yl]acetic acid; |
| B-192 | 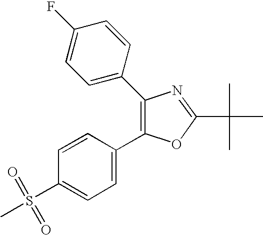 |
| | 2-(tert-butyl)-4-(4-fluorophenyl)-5-[4- |
| | (methylsulfonyl)phenyl]oxazole; |
| B-193 | 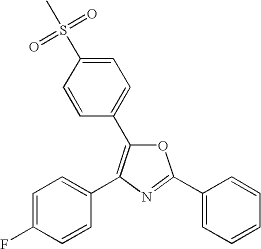 |
| | 4-(4-fluorophenyl)-5-[4-(methylsulfonyl) |
| | phenyl]-2-phenyloxazole; |
| B-194 | 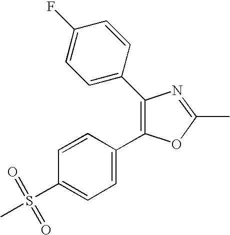 |
| | 4-(4-fluorophenyl)-2-methyl-5-[4-(methyl- |
| | sulfonyl)phenyl]oxazole; |
| B-195 | 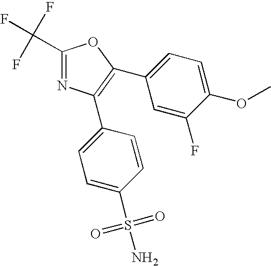 |
| | 4-[5-(3-fluoro-4-methoxyphenyl)-2-trifluoromethyl- |
| | 4-oxazolyl]benzenesulfonamide; |
| B-196 | 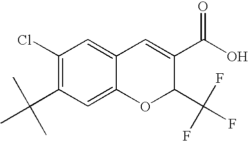 |
| | 6-chloro-7-(1,1-dimethylethyl)-2-trifluoromethyl-2H- |
| | 1-benzopyran-3-carboxylic acid; |
| B-197 | 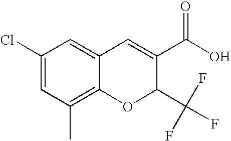 |
| | 6-chloro-8-methyl-2-trifluoromethyl-2H-1-benzopyran- |
| | 3-carboxylic acid; |
| B-198 | 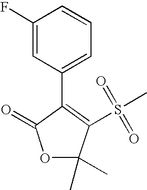 |
| | 5,5-dimethyl-3-(3-fluorophenyl)-4-methylsulfonyl- |
| | 2(5H)-furanone; |
| B-199 | 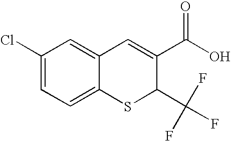 |
| | 6-chloro-2-trifluoromethyl-2H-1-benzothiopyran-3- |
| | carboxylic acid; |
| B-200 | 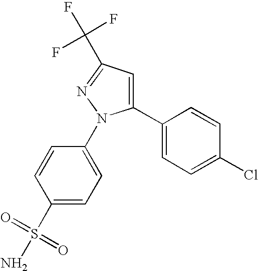 |
| | 4-[5-(4-chlorophenyl)-3-(trifluoramethyl)-1H- |
| | pyrazol-1-yl]benzenesulfonamide; |
| B-201 | 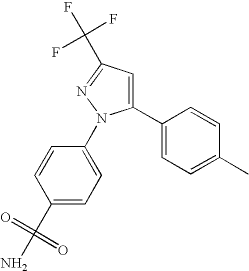 |
| | 4-[5-(4-methylphenyl)-3-(trifluoromethyl)-1H- |
| | pyrazol-1-yl]benzenesulfonamide; |
| B-202 | 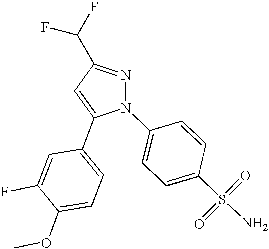 |
| | 4-[5-(3-fluoro-4-methoxyphenyl)-3-(difluoromethyl)- |
| | 1H-pyrazol-1-yl]benzenesulfonamide; |
| B-203 | 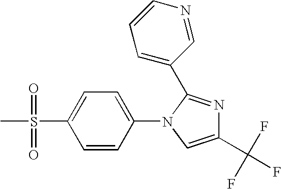 |
| | 3-[1-[4-(methylsulfonyl)phenyl]-4-trifluoromethyl- |
| | 1H-imidazal-2-yl]pyridine; |
| B-204 | 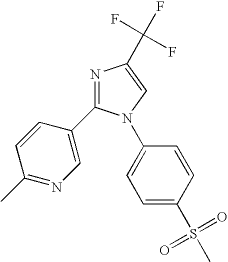 |
| | 2-methyl-5-[1-[4-(methylsulfonyl)phenyl]-4- |
| | trifluoromethyl-1H-imidazol-2-yl]pyridine; |
| B-205 | 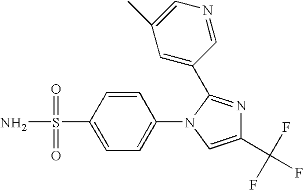 |
| | 4-[2-(5-methylpyridin-3-yl)-4-(trifluoromethyl)- |
| | 1H-imidazol-1-yl]benzenesulfonamide; |
| B-206 | 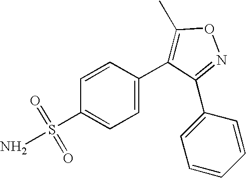 |
| | 4-[5-methyl-3-phenylisoxazol-4-yl]benzenesulfonamide; |
| B-207 | 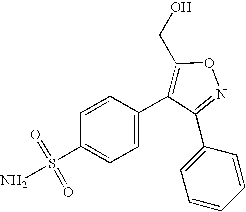 |
| | 4-[5-hydroxymethyl-3-phenylisoxazol-4- |
| | yl]benzenesulfonamide; |
| B-208 | 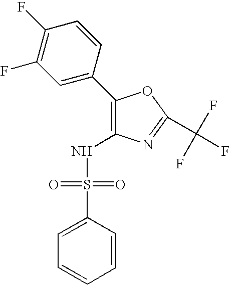 |
| | [2-trifluoromethyl-5-(3,4-difluorophenyl)-4- |
| | oxazolyl]benzenesulfonamide; |
| B-209 | 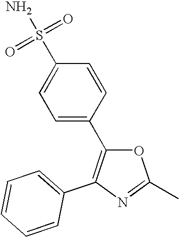 |
| | 2-methyl-4-phenyl-5-oxazolyl]benzenesulfonamide; |
| B-210 | 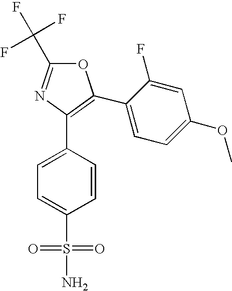 |
| | 4-[5-(2-fluoro-4-methoxyphenyl)-2-trifluoromethyl- |
| | 4-oxazolyl]benzenesulfonamide; |
| B-211 | 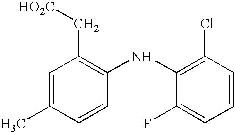 |
| B-212 | 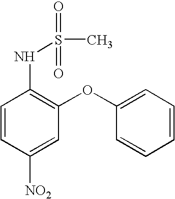 |
| | N-(4-nitro-2-phenoxy-phenyl)-methanesulfonamide |
| B-213 | 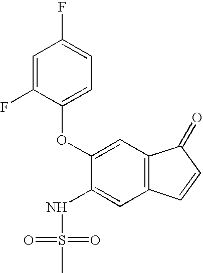 |
| | N-[6-(2,4-difluoro-phenoxy)-1-oxo-inden-5-yl]- |
| | methanesulfonamide |
| B-214 | 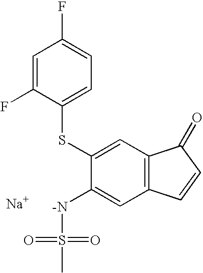 |
| | N-[6-(2,4-difluoro-phenylsulfanyl)-1-oxo-1H-inden- |
| | 5-yl]-methanesulfonamide, soldium salt |
| B-215 | 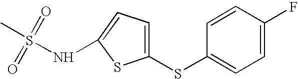 |
| | N-[5-(4-fluoro-phenylsulfanyl)-thiophen-2- |
| | yl]-methanesulfonamide |
| B-216 | 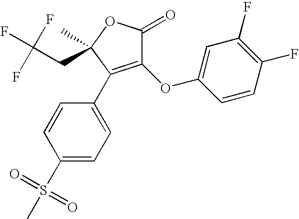 |
| | 3-(3,4-difluoro-phenoxy)-4-(4-methanesulfonyl- |
| | phenyl)-5-methyl-5-(2,2,2-trifluoro-ethyl)- |
| | 5H-furan-2-one |
| B-217 | 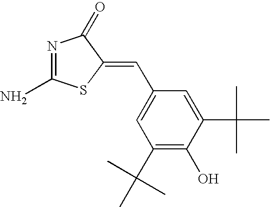 |
| | (5Z)-2-amino-5-[[3,5-bis(1,1-dimethylethyl)-4- |
| | hydroxyphenyl]methylene]-4(5H)-thiazolone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 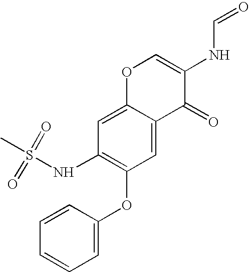 |
| | N-[3-(formylamino)-4-oxo-6-phenoxy-4H-1- |
| | benzopyran-7-yl]-methanesulfonamide |
| B-225 | D-1367 |
| B-226 | L-748731 |
| B-227 | 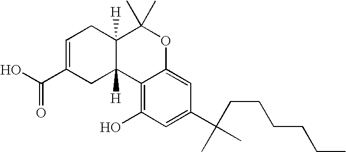 |
| | (6aR,10aR)-3-(1,1-dimethylheptyl)-6a,7,10,10a- |
| | tetrahydro-]-hydroxy-6,6-dimiethyl-6H- |
| | dibenzo[b,d]pyran-9-carboxylic acid |
| B-228 | CGP-28238 |
| B-229 | 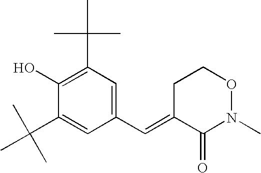 |
| | 4-[[3,5-bis(1,1-dimethylethyl)-4- |
| | hydroxyphenyl]methylene]dihydro-2- |
| | methyl-2H-1,2-oxazin-3(4H)-one |
| B-230 | GR-253035 |
| B-231 | 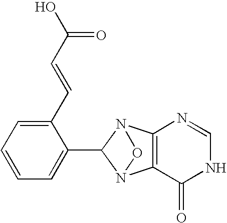 |
| | 2-(6-dioxo-9H-purin-8-yl)cinnamic acid |
| B-232 | S-2474 |
| B-233 | 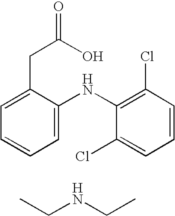 |
| B-234 | 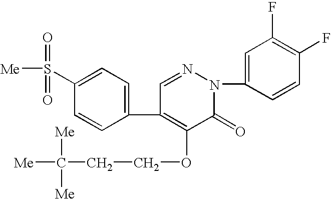 |
| B-235 | 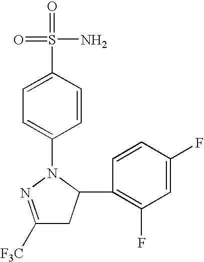 |
| B-236 | 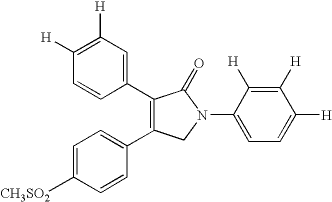 |
| B-237 | 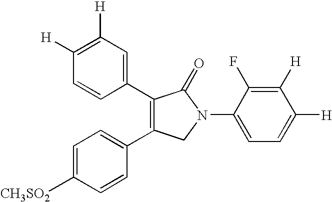 |
| B-238 | 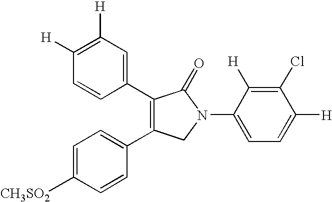 |
| B-239 | 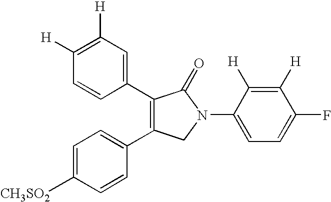 |
| B-240 | 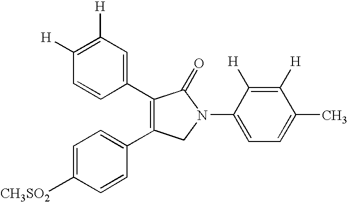 |
| B-241 | 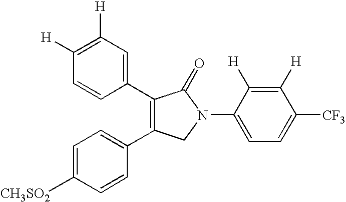 |
| B-242 | 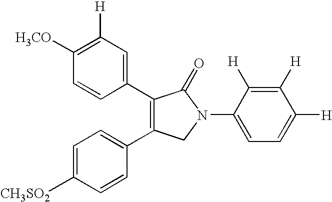 |
| B-243 | 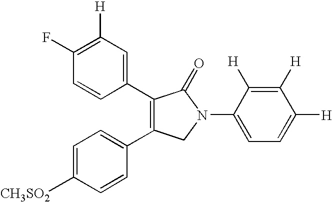 |
| B-244 | 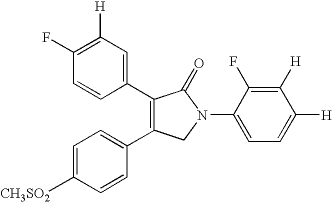 |
| B-245 | 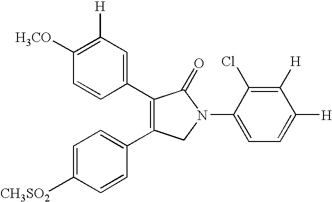 |
| B-246 | 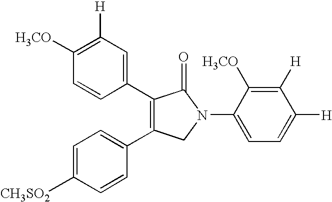 |
| B-247 | 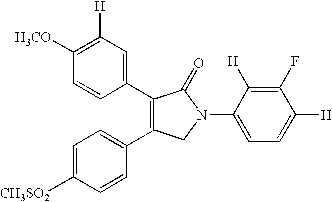 |
| B-248 | 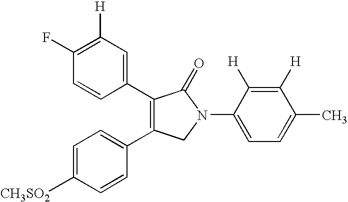 |
| B-249 | 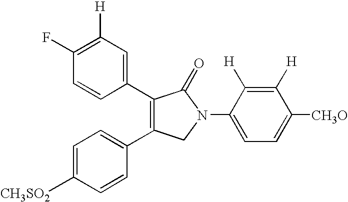 |
| B-250 | 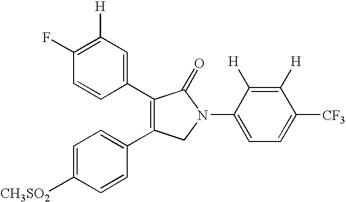 |
| B-251 | 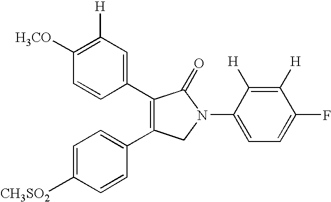 |
| B-252 | 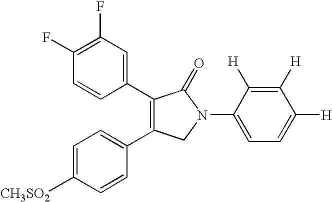 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R— and S-enantiomers, diastereomers, d-isomers, I-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day.kg, and even more typically, from about 0.18 to about 0.4 mg/day-kg.

In still another embodiment, when the cyclooxygenase-2 selective inhibitor comprises etoricoxib, it is typical that the amount used is within a range of from about 0.5 to about 5 mg/day. kg, and even more typically, from about 0.8 to about 4 mg/day.kg.

Further, when the cyclooxygenase-2 selective inhibitor comprises celecoxib, it is typical that the amount used is within a range of from about 1 to about 20 mg/day. kg, even more typically, from about 1.4 to about 8.6 mg/day.kg, and yet more typically, from about 2 to about 3 mg/day.kg.

When the cyclooxygenase-2 selective inhibitor comprises valdecoxib, it is typical that the amount used is within a range of from about 0.1 to about 5 mg/day.kg, and even more typically, from about 0.8 to about 4 mg/day.kg.

In a further embodiment, when the cyclooxygenase-2 selective inhibitor comprises parecoxib, it is typical that the amount used is within a range of from about 0.1 to about 5 mg/day.kg, and even more typically, from about 1 to about 3 mg/day.kg.

Those skilled in the art will appreciate that dosages may also be determined with guidance from Goodman & Goldman's The Pharmacological Basis of Therapeutics, Ninth Edition (1996), Appendix II, pp.1707-1711 and from Goodman & Goldman's The Pharmacological Basis of Therapeutics, Tenth Edition (2001), Appendix II, pp. 475-493.

Serotonin Modulating Agents

In addition to a cyclooxygenase-2 selective inhibitor, the composition of the invention also comprises a therapeutically effective amount of a serotonin modulating agent or an isomer, ester, a pharmaceutically acceptable salt or a prodrug thereof. A number of different serotonin modulating agents are suitable for use in the present invention. In some aspects, the serotonin modulating agent may be a serotonin receptor antagonist. In other aspects, the serotonin modulating agent may be a serotonin receptor agonist. In still further aspects, the serotonin modulating agent may be a serotonin reuptake inhibitor.

In one aspect of the invention, the serotonin modulating agent is a serotonin receptor antagonist. In one embodiment, the serotonin receptor antagonist is a 5-HT₁ antagonist. In one alternative of this embodiment, the 5-HT₁ antagonist is selected from the group consisting of:
3-[4-(4-chlorophenyl)piperazin-1-yl]-1,1-diphenyl-2-propanol;
4-[3-[t-butylamino]-2-hydroxypropoxy]-1H-indole-2-carbonitrile;
3-[3-(dimethylamino)propyl]-4-hydroxy-N-[4-(4-pyridinyl)phenyl]benzamide;
N-[4-methoxy-3-(4-methyl-1-piperazinyl)phenyl]-2′-methyl-4′-(5-methyl-1,2,4-oxadiazol-3-yl)-1,1′-biphenyl-4-carboxamide hydrochloride;
1-[(1-methylethyl)amino]-3-[2-(1H-pyrrol-1-yl)phenoxy]propan-2-ol;
1-(2-methoxyphenyl)-4-(4-succinimidobutyl)piperazine;
1-(2-methoxyphenyl)-4-(4-phthalimidobutyl)piperazine;
1-(1H-indol-4-yloxy)-3-[(1-methylethyl)amino]-2-propanol;
(S)-1-(1H-indol-4-yloxy)-3-[(1-methylethyl)amino]-2-propanol,
N-[3-[3-(dimethylamino)ethoxy]-4-methoxyphenyl]-2′-methyl-4′-(5-methyl-1,2,4-oxadiazol-3-yl)-[1,1′-biphenyl]-4-carboxamide;
8-[(2,3-dihydro-1,4-benzodioxin-2-yl)methyl]-1-phenyl-1,3,8-triazaspiro[4,5]decan-4-one; and

(S)-N-tert-butyl-3-(4-(2-methoxyphenyl)-piperazin-1-yl)-2-phenylpropanamide, or an isomer, ester, pharmaceutically acceptable salt or prodrug thereof.

In another embodiment, the serotonin receptor antagonist is a 5-HT₂ antagonist. In one alternative of this embodiment, the 5-HT₂ antagonist is selected from the group consisting of:
8-[3-(4-fluorophenoxy)propyl]-1-phenyl-1,3,8-triazaspiro[4.5]-decan-4-one;
N-[2-[[3-(dimethylamino)propyl]thio]phenyl]-3-phenyl-2-propenamide;
4-(5H-dibenzo[a,d]cyclohepten-5-ylidene)-1-methylpiperidine;
8-chloro-11-(1-piperazinyl)-5H-dibenzo[b,e][1,4]diazepine;
4-(4-fluorobenzoyl)-1-(4-phenylbutyl)-piperidine oxalate;
3-[2-[4-(4-fluorobenzoyl)-1-piperidinyl]ethyl]-2,4[1H ,3H]-quinazolinedione;
α-phenyl-1-(2-phenylethyl)-4-piperidinemethanol;
metergoline phenylmethyl ester;
1,2,3,4,10,14b-hexahydro-2-methyldibenzo[c,f]pyrazino[1,2-a]azepine;
8-[5-(2,4-dimethoxy-5-(4-trifluoromethylphenylsulphonamido)phenyl-5-oxopentyl]-1,3,8-triazaspiro[4.5]decane-2,4-dione;
N-(1-methyl-1H-indol-5-yl)-N′-3-pyridinyl-urea;
N-(1-methyl-5-indolyl)-N′-(3-methyl-5-isothiazolyl)urea;
(+)-cis4,5,7a,8,9,10,11,11a-octahydro-7H-10-methylindolo[1,7-bc][2,6]-nahthyridine; and
8-[4-(4-fluorophenyl)-4-oxobutyl]-1-phenyl-1,3,8-triazaspiro[4,5]decan-4-one, or an isomer, ester, pharmaceutically acceptable salt or prodrug thereof.

In yet another embodiment, the serotonin receptor antagonist is a 5-HT₃ antagonist. In one alternative of this embodiment, the 5-HT₃ antagonist is selected from the group consisting of:
3aS-2-[(S)-1-Azabicyclo [2.2.2]oct-3-yl]-2,3,3a,4,5,6-hexahydro-1-oxo-1H-benz[de]isoquinoline hydrochloride (palonosetron);
3-(4-allylpiperazin-1-yl)-2-quinoxalinecarbonitrile maleate;
tropanyl 3,5-dichlorobenzoate;
tropanyl 3,5-dimethylbenzoate; and
N-(1-azabicyclo[2.2.2]oct-3-yl)-6-chloro4-methyl-3-oxo-3,4-dihydro-2H-1,4-benzoxazine-8-carboxamide,
or an isomer, ester, pharmaceutically acceptable salt or prodrug thereof.

In a further embodiment, the serotonin receptor antagonist is a 5-HT₄ antagonist. In one alternative of this embodiment, the 5-HT₄ antagonist is selected from the group consisting of:
1-methyl-1H-indole-3-carboxylic acid;
3-(piperidin-1-yl)propyl-4-amino-5-chloro-2-methoxybenzoate;
1-[4-amino-5-chloro-2-(3,5-dimethoxyphenyl)methyloxy]-3-[1-[2-methylsulphonylamino]ethyl]piperidin-4-yl]propan-1-one; and
1-piperidinylethyl-1H-indole-3-carboxylate,
or an isomer, ester, pharmaceutically acceptable salt or prodrug thereof.

In another embodiment, the serotonin receptor antagonist is a 5-HT₆ antagonist. In one alternative of this embodiment, the 5-HT₆ antagonist is metergoline phenylmethyl ester, or an isomer, ester, pharmaceutically acceptable salt or prodrug thereof.

In yet another embodiment, the serotonin receptor antagonist is a 5-HT₇ antagonist. In one alternative of this embodiment, the 5-HT₇ antagonist is selected from the group consisting of:
metergoline phenylmethyl ester, and
1-[1-[4,4-bis(4-fluorophenyl)butyl]-4-piperidinyl]1,3-dihydro-2H-benzimidazol-2-one,
or an isomer, ester, pharmaceutically acceptable salt or prodrug thereof

In another aspect of the invention, the serotonin modulating agent is a serotonin receptor agonist. In one embodiment, the serotonin receptor agonist is a 5-HT₁ agonist. In one alternative of this embodiment, the 5-HT₁ agonist is selected from the group consisting of:
6-chloro-2-[piperidinyl-4-thio]pyridine;
8-[2-[4-(2-methoxyphenyl)-1-piperazinyl]ethyl]-8-azaspiro[4.5]decane-7,9-dione;
1-[3-(3,4-methylenedioxyphenoxy)propyl]-4-phenylpiperazine;
5-hydroxy-3-(1-methylpiperidin-4-yl)-1H-indole;
8-[4-[4-(2-pyrimidinyl)-1-piperazinyl]butyl]-8-azaspiro[4.5]decane-7,8-dione;
5-carboxamidotryptamine maleate;
7-trifluoromethyl4-(4-methyl-1-piperazinyl)pyrrolo[1,2-a]-quinoxaline;
1,4-dihydro-3-(1,2,3,6-tetrahydro-4-pyridinyl)-5H-pyrrolo[3,2-b]pyridin-5-one;
5-propoxy-3-(1,2,3,6-tetrahydro-4-pyridinyl)-1H-pyrrolo[3,2b]pyridine hydrochloride;
3-[3-(2-dimethylaminoethyl)-1H-indol-5-yl]-N-(4-methoxybenzyl)acrylamide;
8-hydroxy-2-dipropylaminotetralin hydrobromide;
(2R)-(+)-8-hydroxy-2-(di-n-propylamino)tetralin;
(RS)-trans-8-hydroxy-2-[N-n-propyl-N-(3′-iodo-2′-propenyl)amino]tetralin;
2-[5-[3-(4-methylsulphonylamino)benzyl-1,2,4-oxadiazol-5-yl]-1H-indol-3-yl]ethanamine;
8-[2-(1,4-benzodioxan-2-ylmethylamino)ethyl]-8-azaspiro[4.5]decane-7,9-dione;
nonyloxytryptamineoxalate;
5-methoxy-3-(1,2,5,6-tetrahydro-4-pyridinyl)-1H-indole; and
N-(3-trifluoromethylphenyl)piperazine,
or an isomer, ester, pharmaceutically acceptable salt or prodrug thereof.

In another embodiment, the serotonin receptor agonist is a 5-HT₂ agonist. In one alternative of this embodiment, the 5-HT₂ agonist is selected from the group consisting of:
α-methyl-5-(2-thienylmethoxy)-1H-indole-3-ethanamine;
1-(3-chlorophenyl)piperazine;
α-methyl-5-hydroxytryptamine maleate; and
6-chloro-2-(1-piperazinyl)pyrazine,
or an isomer, ester, pharmaceutically acceptable salt or prodrug thereof.

In yet another embodiment, the serotonin receptor agonist is a 5-HT₃ agonist. In one alternative of this embodiment, the 5-HT₃ agonist is selected from the group consisting of:
1-(3-chlorophenyl)biguanide;
2-methyl-5-hydroxytryptamine hydrochloride;
2-(1-N-methylpiperazinyl)quinoline;
1-phenylbiguanide hydrochloride;
2-(1-piperazinyl)quinoline;
(R)-N-(1-azabicylco[2.2.2]oct-3-yl)-2-(1-methyl-1H-indol-3-yl)-2-(1-methyl-1H-indol-3-yl)-2-oxoacetamide; and
1-(6-chloro-2-pyridinyl)-4-piperidinamine,
or an isomer, ester, pharmaceutically acceptable salt or prodrug thereof.

In a further embodiment, the serotonin receptor agonist is a 5-HT₄ agonist. In one alternative of this embodiment, the 5-HT₄ agonist is selected from the group consisting of:
2-[1-(4-piperonyl)piperazinyl]benzothiazole;
1-(4-amino-5-chloro-2-methoxyphenyl)-3-[1-butyl-4-piperidinyl]-1-propanone; and
1-(4-amino-5-chloro-2-methoxyphenyl)-3-[1-2-methylsulphonylamino)ethyl-4-piperidinyl]-1-propanone, or an isomer, ester, pharmaceutically acceptable salt or prodrug thereof.

In a still further embodiment, the serotonin receptor agonist is a 5-HT₅ agonist. In one alternative of this embodiment, the 5-HT₅ agonist is 5-carboxamidotryptamine maleate, or an isomer, ester, pharmaceutically acceptable salt or prodrug thereof.

In another embodiment, the serotonin receptor agonist is a 5-HT₆ agonist. In one alternative of this embodiment, the 5-HT₆ agonist is 2-methyl-5-hydroxytryptamine hydrochloride, or an isomer, ester, pharmaceutically acceptable salt or prodrug thereof.

In yet another embodiment, the serotonin receptor agonist is a 5-HT₇ agonist. In one alternative of this embodiment, the 5-HT₇ agonist is selected from the group consisting of 5-carboxamidotryptamine maleate, and (+,−)-8-hydroxy-2-dipropylaminotetralin, or an isomer, ester, pharmaceutically acceptable salt or prodrug thereof.

In still another embodiment, compounds that are useful for the serotonin modulating agent or a pharmaceutically acceptable salt or prodrug thereof in connection with the present invention, include, but are not limited to:
N-acetyltryptamine;
P-chlorophenylalanine;
1-chloro-11-(4-methyl-1-piperazinyl)-5H-dibenzo[b,e][1,4]diazepine;
(5′α,10α)-9,10-dihydro-12′-hydroxy-2′-(1-methylethyl)-5′-(phenylmethyl)-ergotaman-3′,6′,18-trione;
9,10,dihydro-12′-hydroxy-2′-methyl-5′-(phenylmethyl)ergotaman-3′,6′,18-trione;
1-[10,11-dihydro-8-(methylthio)dibenzo[b,f]thiepin-10-yl]-4-methylpiperazine;
[8β(S)]-9,10-didehydro-N-[1-(hydroxymethyl)propyl]-6-methylergoline-8-carboxamide;
[8β(S)]-9,10-didehydro-N-[1-(hydroxymethyl)propyl]-1,6-dimethylergoline-8-carboxamide;
cis-9-octadecenoamide;
[1aR-(1aR*,4E,7aS*,10aS*,−10bR*)]-2,3-6,7,7a,8,10a,10b-octahydro-1a,5-dimethyl-8-methyleneoxireno[9,10]cyclodeca[1,2-b]furan-9-(1aH)-one; and
N-(3-trifluoromethylphenyl)piperazine.

In a further embodiment, the serotonin modulating agent is selected from the group consisting of compounds having the general Formula I shown below and containing, by way of example and not limitation, the compounds listed below. Furthermore, the serotonin modulating agents useful in the practice of the present invention are described in U.S. Pat. No. 5,436,246 which is herein incorporated by reference in its entirety. 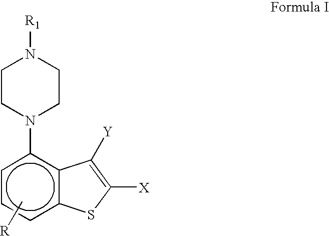
wherein:
Y is hydrogen or C₁₋₃ alkyl;
R is a substituent selected from the group consisting of hydrogen, C₁₋₄ alkyl, C₁₋₄ alkoxy, halogen, —CF₃, —OCF₃, and —OH;
R₁ is hydrogen, cycloalkyl, C₁₋₆ alkyl, phenyl optionally substituted, phenylalkyl, or phenylamidoalkyl;
X is hydrogen, —(CH₂)ₙX₁, —CH═CHX₁ or —CHX₂—(CH₂)_{q}—CH₃;
n is an integer from 0-2;
q is either the integer 0 or 1;
X₁ is —OH, —OR₂, —NR₂R₃, —CO₂R₂, —CONR₂R₃, —CN, CH₂OH or —COR₂;
R₂ and R₃ are each independently hydrogen, C₁₋₄ alkyl, phenyl optionally substituted, phenylalkyl, or R₂ and R₃ together form a (CH₂)ₘ cycloalkyl, wherein m=2-6; and
X₂ is —OR₄ or —NR₄R₅ in which R₄ and R₅ are each independently hydrogen or C₁₋₄ alkyl;
an isomer, ester, pharmaceutically acceptable salt or prodrug thereof;
with the proviso that when n is O or X is —CH═CHX₁, then X₁ is not —OH, —OR₂, or —NR₂ R₃.

Examples of suitable compounds having formula I include:
4-[4-(2-phenylethyl)-1-piperazinyl]-benzo[b]thiophene-2-methanolmonohydrochloride;
4-[4-(2-phenylethyl)-1-piperazinyl]-benzo[b]thiophene-2-carboxamide;
4-[4-(2-phenylethyl)-1-piperazinyl]-benzo[b]thiophene-2-nitrile;
4-[4-(3-phenylpropyl)-1-piperazinyl]-benzo[b]thiophene-2-methanol;
4-[4-(3-phenylpropyl)-1-piperazinyl]-benzo[b]thiophene-2-carboxamide;
4-[4-[2-(4-methoxyphenyl)ethyl]-1-piperazinyl]-benzo[b]thiophene-2-methanol;
4-[4-[2-(4-chlorophenyl)ethyl]-1-piperazinyl]-benzo[b]thiophene-2-carboxamide;
4-[4-[2-(4-chlorophenyl)ethyl]-1-piperazinyl]-benzo[b]thiophene-2-methanol;
4-[4-[2-(4-methylphenyl)ethyl]-1-piperazinyl]-benzo[b]thiophene-2-methanol;
4-[4-(2-phenylethyl)-1-piperazinyl]-benzo[b]thiophene-2-(N-methyl)-carboxamide;
4-[4-(2-phenylethyl)-1-piperazinyl]-benzo[b]thiophene-2-(N,N-dimethyl)-carboxamide;
4-[4-[2-(4-methylphenyl)ethyl]-1-piperazinyl]-benzo[b]thiophene-2-carboxamide;
4-[4-[2-(4-fluorophenyl)ethyl]-1-piperazinyl]-benzo[b]thiophene-2-methanol;
4-[4-[2-(4-fluorophenyl)ethyl]-1-piperazinyl]-benzo[b]thiophene-2-carboxamide;
ethyl-4-[(4-propyl)-1-piperazinyl]benzo[b]thiophene-2-carboxylatehydrochloride;
4-[(4-propyl)-1-piperazinyl]benzo[b]thiophene-2-methanolhydrochloride;
4-[4-(2-phenylethyl)-1-piperazinyl]-benzo[b]thiophene-2-(N-ethyl)carboxamidehydrochloride;
4-[4-(2-phenylethyl)-1-piperazinyl]-benzo[b]thiophene-2-(O-methyl)-methanolhydrochloride;
4-[4-propyl-1-piperazinyl]-benzo[b]thiophene-2-[N-methyl]carboxamidehydrochloride;
4-[4-methyl-1-piperazinyl]-benzo[b]thiophene-2-methanolhydrochloride;
4-[4-(2-phenylethyl)-1-piperazinyl]-benzo[b]thiophene-2-(N-methyl-N-methoxy)-carboxamidehydrochloride;
2-[4-[4-(2-phenylethyl)-1-piperazinyl]benzo[b]thiophene-2-]-(2-propanol)hydrochloride hemihydrate;
1-[4-(4-phenethyl-piperazin-1-yl)-benzo[b]thiophen-2-yl]-ethanonehydrochloride;
1-[4-(4-phenethyl-piperazin-1-yl)-benzo[b]thiophen-2-yl]-ethanolhydrochloride;
4-[4-phenylmethyl-1-piperazinyl]-benzo[b]thiophene-2-methoxymethylhydrochloride;
4-(1-piperazinyl)-benzo[b]thiophene-2-methoxymethylhydrochloride;
4-[4-(2-(4-fluorophenyl)-ethyl)-1-piperazinyl]benzo[b]thiophene-2-methoxymethylhydrochloride;
4-[4-(2-phenylethyl)-1-piperazinly]-benzo[b]thiopene-2-carboxaldehyde;
4-[4-(4-phenylcarbomoyl-butyl)-piperazin-1-yl]-benzo[b]thiopen-2-carboxylicacidethylesterhydrochloride;
4-(1-piperazinyl)benzo[b]thiophene-2-(N-methyl)carboxamide;
44-[2-(4-nitrophenyl)ethyl]-1-piperazinyl]-benzo[b]thiophene-2-methanolhydrochloridedihydrochloride;
4-(1-piperazinyl)benzo[b]thiophene-2-methanolhydrochloride;
ethyl4-[4-[2-(4-nitrophenyl)ethyl]-1-piperazinyl-benzo[b]thiophene-2-carboxylatehydrochloride;
5-[4-(2-Hydroxymethyl-benzo[b]thiophen4-yl)-piperazin-1-yl)-pentanoicacidphenylamidehydrochloride;
2-[4-(4-phenethyl-piperazin-1-yl)-benzo[b]thiophen-2-ylmethyl]-isoindole-1,3-dionehydrochloride;
4-[4-(2-phenylethyl)-1-piperazinyl]-benzo[b]thiophene-2-methanaminedihydrochloride;
[4-(4-phenthyl-piperazin-1-yl)-benzo[b]thiophen-2-yl]-piperidin-1-ylmethanonehydrochloride;
[4-(4-phenethyl-piperazin-1-yl)-benzo[b]thiophen-2-yl]pyrrolidin-1-ylmethanonehydrochloride;
3-[4-(4-phenethyl-piperazin-1-yl)-benzo[b]thiophen-2-yl]-acrylicacidethylesterhydrochloride:yl]-acrylicacidethylesterhydrochloride;
3-[4-(4-phenethyl-piperazin-1-yl)-benzo[b]thiophen-2-yl]-prop-2-en-1-olhydrochloride;
3-[4-(4-phenethyl-piperazin-1-yl)-benzo[b]thiophen-2-yl]-acrylonitrilehydrochloride;
3-[4-(4-phenethyl-piperazin-1-yl)-benzo[b]thiophen-2-yl]-acrylamidehydrochloride;
3-[4-(4-phenethyl-piperazin-1-yl)-benzo[b]thiophen-2-yl]-propionicacidethylesterhydrochloride;
3-[4-(4-phenethyl-piperazin-1-yl)-benzo[b]thiophen-2-yl]-propan-1-olhydrochloride;
3-[4-(4-phenethyl-piperazin-1-yl)-benzo[b]thiophen-2-yl]-propionitrilehydrochloride; and
3-[4-(4-phenethyl-piperazin-1-yl)-benzo[b]thiophen-2-yl]-propionamidehydrochloride.

In a still further embodiment, the serotonin modulating agent is selected from the group consisting of compounds having the general Formula II shown below and containing, by way of example and not limitation, the compounds listed below. Furthermore, the serotonin modulating agents useful in the practice of the present invention are described in U.S. Pat. No. 5,559,143 which is herein incorporated by reference in its entirety. 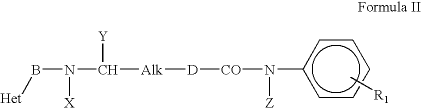
wherein:
B is a C₁₋₄ alkylene bridging group;
Alk is a linear alkylene group containing from 2-8 carbon atoms which may optionally be mono-substituted at one carbon atom with a C₁₋₄ alkyl, phenyl, substituted phenyl or an alkylphenyl substituent in which the phenyl ring may be optionally substituted;
D is a bond or an ethenylene group;
X, Y, and Z are each independently represented by hydrogen, C₁₋₄ alkyl, phenyl, substituted phenyl or alkylphenyl in which the phenyl ring may be optionally substituted;
R¹ is a substituent selected from the group consisting of hydrogen, halogen, C₁₋₄ alkyl, C₁₋₅ alkoxy, —CF₃, —OCF₃, —OH, —NO₂, —CN, —CONR₂R₃, —NR₂R₃, —COOR₄, —OCH₂COOR₄, —CH₂SO₂NR₂R₃, and —SO₂NR₂R₃;
R₂ and R₃ are each independently H or a C₁₋₄ alkyl;
R₄ is H, C₁₋₄ alkyl, phenyl, substituted phenyl or an alkylphenyl substituent in which the phenyl ring may be optionally substituted;
Het is represented by one of the following substituents: 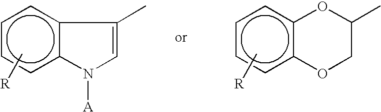
   Wherein:
R is a substituent selected from the group consisting of hydrogen, halogen, C₁₋₄ alkyl, C₁₋₄ alkoxy, —O—CH₂—C₆H₅, —CF₃, —OCF₃, —OH, —NO₂, —CN, —CONR₅R₆, —CH₂SO₂NR₅R₆, —SO₂NR₅R₆, —COOR₇ or —OCH₂COOR₇;
R₅ and R₆ are each independently H or C₁₋₄ alkyl;
R₇ is H, C₁₋₄ alkyl, phenyl, substituted phenyl or an alkylphenyl substituent in which the phenyl ring may be optionally substituted; and
A is H, or C₁₋₄ alkyl;
or an isomer, ester, pharmaceutically acceptable salt or prodrug thereof.;
with the proviso that when Her is an indolyl derivative, then R₁ is not a carbonyl derivative.

Examples of suitable compounds having formula II include:
6-[[2-(5-hydroxy-1H-indol-3-yl)ethyl]amino]-N-[(4-(trifluoromethyl)phenyl]-heptanamide;
7-[[2-(5-hydroxy-1H-indol-3-yl)ethyl]amino]-N-(4-methoxyphenyl)-octanamide;
6-[[2-(5-hydroxy-1H-indol-3-yl)ethyl]amino]-N-phenylheptanamide;
5-[[2-(5-hydroxy-1H-indol-3-yl)ethyl]amino]-N-[4-(trifluoromethyl)phenyl]-hexanamide;
6-[[2-(5-hydroxy-1H-indol-3-yl)ethyl]amino]-N-(4-methoxyphenyl)-heptanamide;
4-[[2-(5-hydroxy-1H-indol-3-yl)ethyl]amino]-N-[4-(trifluoromethyl)phenyl]-pentanamide;
6-[[2-(5-methoxy-1H-indol-3-yl)ethyl]amino]-N-[4-(trifluoromethyl)phenyl]-heptanamide;
6-[[2-(5-hydroxy-1H-indol-3-yl)ethyl]methylamino]-N-[4-(trifluoromethyl)phenyl]-heptanamide;
6-[[2-(5-hydroxy-1H-indol-3-yl)ethyl]amino]-N-(2-methoxyphenyl)-heptanamide;
6-[[2-(5-carboxamido-1H-indol-3-yl)ethyl]amino]-N-(4-methoxyphenyl)-heptanamide;
6-[[2-(1H-indol-3-yl)ethyl]amino]-N-[4-(trifluoromethyl)phenyl]-hexanamide;
6-[[2-(5-hydroxy-1H-indol-3-yl)ethyl]amino]-N-[4-(1propyl)phenyl]-hexanamide;
5-[[2-(5-hydroxy-1H-indol-3-yl)ethyl]amino]-N-[4-(1propyloxy)phenyl]-hexanamide;
6-[2-[(2,3-dihydro-1,4-benzodioxin-2-yl)ethyl]amino]-N-phenyl-hexanamide;
6-[(2,3-dihydro-1,4-benzodioxin-2-yl)methylamino]-N-[4-(trifluoromethyl)phenyl]-heptanamide;
6-[(2,3-dihydro-1,4-benzodioxin-2-yl)methylamino]-N-(4-methoxyphenyl)-heptanamide;
6-[[(2,3-dihydro-1,4-benzodioxin-2-yl)methyl]-methylamino]-N-[4-(trifluoromethyl)phenyl]-hexanamide;
6-[(2,3-dihydro-1,4-benzodioxin-2-yl)methylamino]-N-[2-trifluoromethyl)phenyl]-hexanamide;
7-[[2-(5-hydroxy-1H-indol-3-yl)ethyl]amino]-N-(4-methoxyphenyl)-heptanamide;
7-[[2-(5-hydroxy-1H-indol-3-yl)ethyl]amino]-N-(2-methoxyphenyl)-heptanamide;
6-[[2-(4-hydroxy-1H-indol-3-yl)ethyl]amino]-N-(4-methoxyphenyl)-heptanamide;
6-[[2-(5-chloro-1H-indol-3-yl)ethyl]amino]-N-(4-methoxyphenyl)-heptanamide;
7-[[2-(5-hydroxy-1H-indol-3-yl)ethyl]amino]-N-(3-methoxyphenyl)-octanamide;
6-[[2-(5-hydroxy-1H-indol-3-yl)ethyl]amino]-N-[2-(trifluoromethyl)phenyl]-hexanamide;
6-[[2-(5-hydroxy-1H-indol-3-yl)ethyl]amino]-N-[3-(trifluoromethyl)phenyl]-hexanamide;
6-[[2-(5-hydroxy-1H-indol-3-yl)ethyl]amino]-4-methyl-N-(4-methoxyphenyl)-hexanamide;
6-[[3-(5-hydroxy-1H-indol-3-yl)propyl]amino]-N-(4-methoxyphenyl)-hexanamide;6-[[2-(5-hydroxy-1H-indol-3-yl)ethyl]amino]-N-(3-methoxyphenyl)-hexanamide;
6-[[2-(5-hydroxy-1-methyl-indol-3-yl)ethyl]amino]-N-(4-methoxyphenyl)-hexanamide;
6-[(2,3-dihydro-8-methoxy-1,4-benzodioxin-2-yl)methylamino]-N-(4-methoxyphenyl)-hexanamide;
5-[(2,3-dihydro-1,4-benzodioxin-2-yl)methylamino]-N-[4(trifluoromethyl)phenyl]-pentanamide;
4-[(2,3-dihydro-1,4-benzodioxin-2-yl)methylamino]-N-(4-methoxyphenyl)-butanamide;
7-[[2-(5-methoxy-1H-indol-3-yl)ethyl]-methylamino]-N-(4-methoxyphenyl)-octanamide;
6-[[2-(5-hydroxy-1H-indol-3-yl)ethyl]amino]-N-(4-methoxyphenyl)-2-hexenamide; and
7-[(2,3-Dihydro-1,4-benzodioxin-2-yl)methylamino]-N-[4-trifluoromethyl)phenyl]-heptanamide.

In yet another aspect of the invention, the serotonin modulating agent is a serotonin reuptake inhibitor. In one embodiment, the serotonin reuptake inhibitor is citalopram (marketed under the trademark Celexa® by Forest Laboratories, Parke-Davis, Inc). In another embodiment, the serotonin reuptake inhibitor is fluoxetine (marketed under the trademark Prozac® by Eli Lilly and Company). In still another embodiment, the serotonin reuptake inhibitor is fluvoxamine (marketed under the trademark Luvox® by Solvay Pharmaceuticals, Inc.). In yet another embodiment, the serotonin reuptake inhibitor is paroxetine (marketed under the trademark Paxil® by SmithKline Beecham Pharmaceuticals, Inc.). In a further embodiment, the serotonin reuptake inhibitor is escitalopram oxalate (marketed under the trademark Lexapro® by Forest Laboratories, Parke-Davis, Inc). In still another embodiment, the serotonin reuptake inhibitor is sertraline (marketed under the trademark Zoloft® by Pfizer, Inc.).

It is also contemplated that a number of suitable metabolites of a serotonin reuptake inhibitor may also be employed in the current invention. By way of example, in one embodiment, the metabolite is nortluoxetine, which is an active metabolite of fluoxetine. By way of further example, in another embodiment, the metabolite is N-demethylsertraline, which is an active metabolite of sertraline.

Generally speaking, the pharmacokinetics of the particular agent to be administered will dictate the most preferred method of administration and dosing regiment. The serotonin modulating agent can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution); alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the serotonin modulating agent can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the serotonin modulating agent can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp.197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. Dosages for a particular individual subject can be determined by one of ordinary skill in the art using conventional considerations. But in general, the amount of serotonin modulating agent will be between about 10 to about 2500 milligrams per day. The daily dose can be administered in one to four doses per day.

In one embodiment, when the serotonin modulating agent comprises sertraline, typically the amount administered is within a range of from about 0.5 to about 200 milligrams per day, and even more typically, between about 50 to about 100 milligrams per day.

In another embodiment, when the serotonin modulating agent is fluvoxamine, typically the amount administered is within a range of from about 0.5 to about 500 milligrams per day, and even more typically, between about 100 to about 300 milligrams per day.

In yet another embodiment, when the serotonin modulating agent is fluoxetine, generally the amount administered is within a range of from about 0.5 to about 150 milligrams per day, and even more typically, between about 20 to about 80 milligrams per day.

In still another embodiment, when the serotonin modulating agent is paroxetine, typically the amount administered is within a range of from about 0.5 to about 100 milligrams per day, and even more typically, between about 10 to about 50 milligrams per day.

In yet a further embodiment, when the serotonin modulating agent is citalopram, typically the amount administered is within a range of from about 0.5 to about 100 milligrams per day, and even more typically, between about 20 to about 40 milligrams per day.

In still another embodiment, when the serotonin modulating agent is escitalopram oxalate, typically the amount administered is within a range of from about 0.5 to about 50 milligrams per day, and even more typically, between about 5 to about 20 milligrams per day. In general, the timing of the administration of the cyclooxygenase-2 selective inhibitor in relation to the administration of the serotonin modulating agent may also vary from subject to subject. In one embodiment, the cyclooxygenase-2 selective inhibitor and serotonin modulating agent may be administered substantially simultaneously, meaning that both agents may be administered to the subject at approximately the same time. For example, the cyclooxygenase-2 selective is administered during a continuous period beginning on the same day as the beginning of the serotonin modulating agent and extending to a period after the end of the serotonin modulating agent. Alternatively, the cyclooxygenase-2 selective inhibitor and serotonin modulating agent may be administered sequentially, meaning that they are administered at separate times during separate treatments. In one embodiment, for example, the cyclooxygenase-2 selective inhibitor is administered during a continuous period beginning prior to administration of the serotonin modulating agent and ending after administration of the serotonin modulating agent. Of course, it is also possible that the cyclooxygenase-2 selective inhibitor may be administered either more or less frequently than the serotonin modulating agent. Moreover, it will be apparent to those skilled in the art that it is possible, and perhaps desirable, to combine various times and methods of administration in the practice of the present invention.

Combination Therapies

Generally speaking, it is contemplated that the composition employed in the practice of the invention may include one or more of any of the cyclooxygenase-2 selective inhibitors detailed above in combination with one or more of any of the serotonin modulating agents detailed above. By way of a non-limiting example, Table 4a details a number of suitable combinations that are useful in the methods and compositions of the current invention. The combination may also include an isomer, a pharmaceutically acceptable salt, ester, or prodrug of any of the cyclooxygenase-2 selective inhibitors and/or serotonin modulating agents listed in Table 4a.

TABLE 4a

| Cyclooxygenase-2 Selective | |
|---|---|
| Inhibitor | Serotonin Modulating Agents |
| a compound having formula I | Citalopram |
| a compound having formula I | Fluoxetine |
| a compound having formula I | Fluvoxamine |
| a compound having formula I | Paroxetine |
| a compound having formula I | Escitalopram oxalate |
| a compound having formula I | Sertraline |
| a compound having formula I | Palonosetron |
| a compound having formula I | Norfluoxetine |
| a compound having formula I | N-demethylsertraline |
| a compound having formula II | Citalopram |
| a compound having formula II | Fluoxetine |
| a compound having formula II | Fluvoxamine |
| a compound having formula II | Paroxetine |
| a compound having formula II | Escitalopram oxalate |
| a compound having formula II | Sertraline |
| a compound having formula II | Palonosetron |
| a compound having formula II | Norfluoxetine |
| a compound having formula II | N-demethylsertraline |
| a compound having formula III | Citalopram |
| a compound having formula III | Fluoxetine |
| a compound having formula III | Fluvoxamine |
| a compound having formula III | Paroxetine |
| a compound having formula III | Escitalopram oxalate |
| a compound having formula III | Sertraline |
| a compound having formula III | Palonosetron |
| a compound having formula III | Norfluoxetine |
| a compound having formula III | N-demethylsertraline |
| a compound having formula IV | Citalopram |
| a compound having formula IV | Fluoxetine |
| a compound having formula IV | Fluvoxamine |
| a compound having formula IV | Paroxetine |
| a compound having formula IV | Escitalopram oxalate |
| a compound having formula IV | Sertraline |
| a compound having formula IV | Palonosetron |
| a compound having formula IV | Norfluoxetine |
| a compound having formula IV | N-demethylsertraline |
| a compound having formula V | Citalopram |
| a compound having formula V | Fluoxetine |
| a compound having formula V | Fluvoxamine |
| a compound having formula V | Paroxetine |
| a compound having formula V | Escitalopram oxalate |
| a compound having formula V | Sertraline |
| a compound having formula V | Palonosetron |
| a compound having formula V | Norfluoxetine |
| a compound having formula V | N-demethylsertraline |

By way of further example, Table 4b details a number of suitable combinations that may be employed in the methods and compositions of the present invention. The combination may also include an isomer, a pharmaceutically acceptable salt, ester, or prodrug of any of the cyclooxygenase-2 selective inhibitors and/or serotonin modulating agents listed in Table 4b.

TABLE 4b

| | |
|---|---|
| a compound selected from the group consisting | Citalopram |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Fluoxetine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Fluvoxamine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Paroxetine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Escitalopram oxalate |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Sertraline |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Palonosetron |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | Norfluoxetine |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |
| a compound selected from the group consisting | N-demethylsertraline |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B23a, B-24, | |
| B-25, B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, | |
| B-236, B-237, B-238, B-239, B-240, B-241, | |
| B-242, B-243 B-244, B-245, B-246, B-247, | |
| B-248, B-249, B-250, B-251, and B-252. | |

By way of yet further example, Table 4c details additional suitable combinations that may be employed in the methods and compositions of the current invention. The combination may also include an isomer, a pharmaceutically acceptable salt, ester, or prodrug of any of the cyclooxygenase-2 selective inhibitors and/or serotonin modulating agents listed in Table 4c.

TABLE 4c

| Cyclooxygenase-2 | |
|---|---|
| Selective Inhibitor | Serotonin Modulating Agents |
| Celecoxib | Citalopram |
| Celecoxib | Fluoxetine |
| Celecoxib | Fluvoxamine |
| Celecoxib | Paroxetine |
| Celecoxib | Escitalopram oxalate |
| Celecoxib | Sertraline |
| Celecoxib | Palonosetron |
| Celecoxib | Norfluoxetine |
| Celecoxib | N-demethylsertraline |
| Cimicoxib | Citalopram |
| Cimicoxib | Fluoxetine |
| Cimicoxib | Fluvoxamine |
| Cimicoxib | Paroxetine |
| Cimicoxib | Escitalopram oxalate |
| Cimicoxib | Sertraline |
| Cimicoxib | Palonosetron |
| Cimicoxib | Norfluoxetine |
| Cimicoxib | N-demethylsertraline |
| Deracoxib | Citalopram |
| Deracoxib | Fluoxetine |
| Deracoxib | Fluvoxamine |
| Deracoxib | Paroxetine |
| Deracoxib | Escitalopram oxalate |
| Deracoxib | Sertraline |
| Deracoxib | Palonosetron |
| Deracoxib | Norfluoxetine |
| Deracoxib | N-demethylsertraline |
| Valdecoxib | Citalopram |
| Valdecoxib | Fluoxetine |
| Valdecoxib | Fluvoxamine |
| Valdecoxib | Paroxetine |
| Valdecoxib | Escitalopram oxalate |
| Valdecoxib | Sertraline |
| Valdecoxib | Palonosetron |
| Valdecoxib | Norfluoxetine |
| Valdecoxib | N-demethylsertraline |
| Rofecoxib | Citalopram |
| Rofecoxib | Fluoxetine |
| Rofecoxib | Fluvoxamine |
| Rofecoxib | Paroxetine |
| Rofecoxib | Escitalopram oxalate |
| Rofecoxib | Sertraline |
| Rofecoxib | Palonosetron |
| Rofecoxib | Norfluoxetine |
| Rofecoxib | N-demethylsertraline |
| Etoricoxib | Citalopram |
| Etoricoxib | Fluoxetine |
| Etoricoxib | Fluvoxamine |
| Etoricoxib | Paroxetine |
| Etoricoxib | Escitalopram oxalate |
| Etoricoxib | Sertraline |
| Etoricoxib | Palonosetron |
| Etoricoxib | Norfluoxetine |
| Etoricoxib | N-demethylsertraline |
| Meloxicam | Citalopram |
| Meloxicam | Fluoxetine |
| Meloxicam | Fluvoxamine |
| Meloxicam | Paroxetine |
| Meloxicam | Escitalopram oxalate |
| Meloxicam | Sertraline |
| Meloxicam | Palonosetron |
| Meloxicam | Norfluoxetine |
| Meloxicam | N-demethylsertraline |
| Parecoxib | Citalopram |
| Parecoxib | Fluoxetine |
| Parecoxib | Fluvoxamine |
| Parecoxib | Paroxetine |
| Parecoxib | Escitalopram oxalate |
| Parecoxib | Sertraline |
| Parecoxib | Palonosetron |
| Parecoxib | Norfluoxetine |
| Parecoxib | N-demethylsertraline |
| 4-(4-cyclohexyl-2- | Citalopram |
| methyloxazol-5-yl)-2- | |
| fluorobenzenesulfonamide | |
| 4-(4-cyclohexyl-2- | Fluoxetine |
| methyloxazol-5-yl)-2- | |
| fluorobenzenesulfonamide | |
| 4-(4-cyclohexyl-2- | Fluvoxamine |
| methyloxazol-5-yl)-2- | |
| fluorobenzenesulfonamide | |
| 4-(4-cyclohexyl-2- | Paroxetine |
| methyloxazol-5-yl)-2- | |
| fluorobenzenesulfonamide | |
| 4-(4-cyclohexyl-2- | Escitalopram oxalate |
| methyloxazol-5-yl)-2- | |
| fluorobenzenesulfonamide | |
| 4-(4-cyclohexyl-2- | Sertraline |
| methyloxazol-5-yl)-2- | |
| fluorobenzenesulfonamide | |
| 4-(4-cyclohexyl-2- | Palonosetron |
| methyloxazol-5-yl)-2- | |
| fluorobenzenesulfonamide | |
| 4-(4-cyclohexyl-2- | Norfluoxetine |
| methyloxazol-5-yl)-2- | |
| fluorobenzenesulfonamide | |
| 4-(4-cyclohexyl-2- | N-demethylsertraline |
| methyloxazol-5-yl)-2- | |
| fluorobenzenesulfonamide | |
| 2-(3,5-difluorophenyl)-3-(4- | Citalopram |
| (methylsulfonyl)phenyl)-2- | |
| cyclopenten-1-one | |
| 2-(3,5-difluorophenyl)-3-(4- | Fluoxetine |
| (methylsulfonyl)phenyl)-2- | |
| cyclopenten-1-one | |
| 2-(3,5-difluorophenyl)-3-(4- | Fluvoxamine |
| (methylsulfonyl)phenyl)-2- | |
| cyclopenten-1-one | |
| Cyclooxygenase-2 | Serotonin Modulating Agents |
| Selective Inhibitor | |
| 2-(3,5-difluorophenyl)-3-(4- | Paroxetine |
| (methylsulfonyl)phenyl)-2- | |
| cyclopenten-1-one | |
| 2-(3,5-difluorophenyl)-3-(4- | Escitalopram oxalate |
| (methylsulfonyl)phenyl)-2- | |
| cyclopenten-1-one | |
| 2-(3,5-difluorophenyl)-3-(4- | Sertraline |
| (methylsulfonyl)phenyl)-2- | |
| cyclopenten-1-one | |
| 2-(3,5-difluorophenyl)-3-(4- | Palonosetron |
| (methylsulfonyl)phenyl)-2- | |
| cyclopenten-1-one | |
| 2-(3,5-difluorophenyl)-3-(4- | Norfluoxetine |
| (methylsulfonyl)phenyl)-2- | |
| cyclopenten-1-one | |
| 2-(3,5-difluorophenyl)-3-(4- | N-demethylsertraline |
| (methylsulfonyl)phenyl)-2- | |
| cyclopenten-1-one | |
| N-[2-(cyclohexyloxy)-4- | Citalopram |
| nitrophenyl]methanesulfonamide | |
| N-[2-(cyclohexyloxy)-4- | Fluoxetine |
| nitrophenyl]methanesulfonamide | |
| N-[2-(cyclohexyloxy)-4- | Fluvoxamine |
| nitrophenyl]methanesulfonamide | |
| N-[2-(cyclohexyloxy)-4- | Paroxetine |
| nitrophenyl]methanesulfonamide | |
| N-[2-(cyclohexyloxy)-4- | Escitalopram oxalate |
| nitrophenyl]methanesulfonamide | |
| N-[2-(cyclohexyloxy)-4- | Sertraline |
| nitrophenyl]methanesulfonamide | |
| N-[2-(cyclohexyloxy)-4- | Palonosetron |
| nitrophenyl]methanesulfonamide | |
| N-[2-(cyclohexyloxy)-4- | Norfluoxetine |
| nitrophenyl]methanesulfonamide | |
| N-[2-(cyclohexyloxy)-4- | N-demethylsertraline |
| nitrophenyl]methanesulfonamide | |
| 2-(3,4-difluorophenyl)-4-(3- | Citalopram |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Fluoxetine |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Fluvoxamine |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Paroxetine |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Escitalopram oxalate |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Sertraline |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Palonosetron |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | Norfluoxetine |
| hydroxy-3-methylbutoxy)- | |
| 5-[4- | |
| (methylsulfonyl)phenyl]- | |
| 3(2H)-pyridazinone | |
| 2-(3,4-difluorophenyl)-4-(3- | N-demethylsertraline |
| hydroxy-3-methylbutoxy)- | |
| 5-[4- | |
| (methylsulfonyl)phenyl]- | |
| 3(2H)-pyridazinone | |
| 2-[(2,4-dichloro-6- | Citalopram |
| methylphenyl)amino]-5- | |
| ethyl-benzeneacetic acid | |
| 2-[(2,4-dichloro-6- | Fluoxetine |
| methylphenyl)amino]-5- | |
| ethyl-benzeneacetic acid | |
| 2-[(2,4-dichloro-6- | Fluvoxamine |
| methylphenyl)amino]-5- | |
| ethyl-benzeneacetic acid | |
| 2-[(2,4-dichloro-6- | Paroxetine |
| methylphenyl)amino]-5- | |
| ethyl-benzeneacetic acid | |
| 2-[(2,4-dichloro-6- | Escitalopram oxalate |
| methylphenyl)amino]-5- | |
| ethyl-benzeneacetic acid | |
| 2-[(2,4-dichloro-6- | Sertraline |
| methylphenyl)amino]-5- | |
| ethyl-benzeneacetic acid | |
| 2-[(2,4-dichloro-6- | Palonosetron |
| methylphenyl)amino]-5- | |
| ethyl-benzeneacetic acid | |
| 2-[(2,4-dichloro-6- | Norfluoxetine |
| methylphenyl)amino]-5- | |
| ethyl-benzeneacetic acid | |
| 2-[(2,4-dichloro-6- | N-demethylsertraline |
| methylphenyl)amino]-5- | |
| ethyl-benzeneacetic acid | |
| (3Z)-3-[(4-chlorophenyl)[4- | Citalopram |
| (methylsulfonyl)phenyl]met | |
| hylene]dihydro-2(3H)- | |
| furanone | |
| (3Z)-3-[(4-chlorophenyl)[4- | Fluoxetine |
| (methylsulfonyl)phenyl]met | |
| hylene]dihydro-2(3H)- | |
| furanone | |
| (3Z)-3-[(4-chlorophenyl)[4- | Fluvoxamine |
| (methylsulfonyl)phenyl]met | |
| hylene]dihydro-2(3H)- | |
| furanone | |
| (3Z)-3-[(4-chlorophenyl)[4- | Paroxetine |
| (methylsulfonyl)phenyl]met | |
| hylene]dihydro-2(3H)- | |
| furanone | |
| (3Z)-3-[(4-chlorophenyl)[4- | Escitalopram oxalate |
| (methylsulfonyl)phenyl]met | |
| hylene]dihydro-2(3H)- | |
| furanone | |
| (3Z)-3-[(4-chlorophenyl)[4- | Sertraline |
| (methylsulfonyl)phenyl]met | |
| hylene]dihydro-2(3H)- | |
| furanone | |
| (3Z)-3-[(4-chlorophenyl)[4- | Palonosetron |
| (methylsulfonyl)phenyl]met | |
| hylene]dihydro-2(3H)- | |
| furanone | |
| (3Z)-3-[(4-chlorophenyl)[4- | Norfluoxetine |
| (methylsulfonyl)phenyl]met | |
| hylene]dihydro-2(3H)- | |
| furanone | |
| (3Z)-3-[(4-chlorophenyl)[4- | N-demethylsertraline |
| (methylsulfonyl)phenyl]met | |
| hylene]dihydro-2(3H)- | |
| furanone | |
| (S)-6,8-dichloro-2- | Citalopram |
| (trifluoromethyl)-2H-1- | |
| benzopyran-3-carboxylic | |
| acid | |
| (S)-6,8-dichloro-2- | Fluoxetine |
| (trifluoromethyl)-2H-1- | |
| benzopyran-3-carboxylic | |
| acid | |
| (S)-6,8-dichloro-2- | Fluvoxamine |
| (trifluoromethyl)-2H-1- | |
| benzopyran-3-carboxylic | |
| acid | |
| (S)-6,8-dichloro-2- | Paroxetine |
| (trifluoromethyl)-2H-1- | |
| benzopyran-3-carboxylic | |
| acid | |
| (S)-6,8-dichloro-2- | Escitalopram oxalate |
| (trifluoromethyl)-2H-1- | |
| benzopyran-3-carboxylic | |
| acid | |
| (S)-6,8-dichloro-2- | Sertraline |
| (trifluoromethyl)-2H-1- | |
| benzopyran-3-carboxylic | |
| acid | |
| (S)-6,8-dichloro-2- | Palonosetron |
| (trifluoromethyl)-2H-1- | |
| benzopyran-3-carboxylic | |
| acid | |
| (S)-6,8-dichloro-2- | Norfluoxetine |
| (trifluoromethyl)-2H-1- | |
| benzopyran-3-carboxylic | |
| acid | |
| (S)-6,8-dichloro-2- | N-demethylsertraline |
| (trifluoromethyl)-2H-1- | |
| benzopyran-3-carboxylic | |
| acid | |
| Lumiracoxib | Citalopram |
| Lumiracoxib | Fluoxetine |
| Lumiracoxib | Fluvoxamine |
| Lumiracoxib | Paroxetine |
| Lumiracoxib | Escitalopram oxalate |
| Lumiracoxib | Sertraline |
| Lumiracoxib | Palonosetron |
| Lumiracoxib | Norfluoxetine |
| Lumiracoxib | N-demethylsertraline |

Indications to be Treated

Generally speaking, the composition comprising a therapeutically effective amount of a cyclooxygenase-2 selective inhibitor and a therapeutically effective amount of a serotonin modulating agent may be employed to treat a number of different types of neoplasia or neoplasia related disorder in a subject irrespective of its stage of progression.

In some aspects, the composition may be administered to either prevent the onset of clinically evident neoplasia altogether or to prevent the onset of a preclinically evident stage of neoplasia in subjects at risk for developing neoplasia. In other aspects, the composition may be administered to prevent the initiation, growth, or spreading of benign cells. In still other aspects, the composition may be administered to prevent the initiation of malignant cells or to arrest or reverse the progression of premalignant cells to malignant cells. In further aspects, the composition may be administered to inhibit neoplasia growth, spreading or metastasis, as well as partial or total destruction of the neoplasia cells. In still further aspects, the serotonin modulating agent may reduce the frequency and severity of nausea associated with chemotherapy treatment.

In one embodiment, the neoplasia is epithelial cell-derived neoplasia (epithelial carcinoma). By way of example, epithelial cell-derived neoplasia includes basal cell carcinoma, squamous cell carcinoma or adenocarcinoma. In another embodiment, the neoplasia is a gastrointestinal cancer. Gastrointestinal cancers include lip cancer, mouth cancer, esophogeal cancer, small bowel cancer, stomach cancer and colon cancer. In still another embodiment, the neoplasia is liver cancer, bladder cancer, pancreas cancer, ovary cancer, cervical cancer, lung cancer, breast cancer and skin cancer, such as squamous cell and basal cell cancers, prostate cancer, brain cancer and renal cell carcinoma. The composition can also be used to treat fibrosis that often occurs with radiation therapy. In yet another embodiment, the composition can be used to treat subjects having adenomatous polyps, including those with familial adenomatous polyposis (FAP).

The cyclooxygenase-2 selective inhibitor and serotonin modulating agent may also be administered with any other drug or agent known in the art to have utility for treating or preventing neoplasia disorders or related diseases. In one embodiment, the antineoplastic agent is an antimetabolite including folate antagonists (e.g. methotrexate), pyrimidine antagonists (e.g. cytarabine, floxuridine, fludarabine, fluorouracil, and gemcitabine), purine antagonists (e.g. cladribine, mercaptopurine, thioguanine), and adenosine deaminase inhibitors (e.g. pentostatin). In an alternative embodiment, the antineoplastic agent is an alkylating agent such as chlorambucil, cyclophosphamide, busulfan, ifosfamide, melphalan, and thiotepa. In yet another embodiment, the antineoplastic agent is an akylator agent such as cisplatin, carboplatin, procarbazine, dacarbazine, and altretamine. In still another embodiment, the antineoplastic agent is an anti-tumor antibiotic such as bleomycin, dactinomycin, and mitomycin. In yet a further embodiment, the antineoplastic agent is an immunological agent such as interferon. In another embodiment, the antineoplastic agent is a plant alkaloid including vinca alkaloids (e.g. vinblastine, vincristine and vinorelbine), epipodophyllotoxins (e.g. etoposide and teniposide), taxanes (e.g. docetaxel and paclitaxel), and camptothecins (e.g. topotecan and irinotecan). Of course those skilled in the art will appreciate that the particular antineoplastic agents to be administered with the composition of the invention will vary considerably depending on the type of neoplasia disorder being treated and its stage of progression.

### EXAMPLES

The following examples are intended to provide illustrations of the application of the present invention. The following examples are not intended to completely define or otherwise limit the scope of the invention.

### Example 1

### Evaluation of COX-1 and COX-2 Activity in Vitro

The COX-2 inhibitors suitable for use in this invention exhibit selective inhibition of COX-1 over COX-2, as measured by IC₅₀ values when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/pg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/pg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### Determining Whether a Composition Reduces Tumor Cell Growth

The ability of a composition of the invention to reduce the growth of tumor cells can readily be determined. As used in the examples, the term “composition” shall include any composition comprising a cyclooxygenase-2 selective inhibitor and serotonin modulating agent detailed herein. By way of example, the cyclooxygenase-2 selective inhibitor utilized for testing the composition may be celecoxib, rofecoxib, valdecoxib, etoricoxib, parecoxib, or deracoxib. The serotonin modulating agent may include fluoxetine, paroxetine, citalopram, escitalopram, or palonosetron. Moreover, various cell lines can be used to determine whether the composition reduces growth of tumor cells. For example, these cell lines include: SW-480 (colonic adenocarcinoma); HT-29 (colonic adenocarcinoma), A-427 (lung adenocarcinoma carcinoma); MCF-7 (breast adenocarcinoma); UACC-375 (melanoma line); and DU-145 (prostrate carcinoma). Cytotoxicity data obtained using these cell lines are indicative of an inhibitory effect on neoplastic lesions. These cell lines are well characterized, and are used by the United States National Cancer Institute in their screening program for new anti-cancer drugs.

By way of illustration, a composition's ability to inhibit tumor cell growth can be measured using the HT-29 human colon carcinoma cell line obtained from ATCC and a SRB assay. HT-29 cells have previously been characterized as a relevant colon tumor cell culture model and may be (Fogh, J., and Trempe, G. In: Human Tumor Cells in Vitro, J. Fogh (eds.), Plenum Press, New York, pp.115-159, 1975). In this assay, HT-29 cells are maintained in RPMI media supplemented with 5% fetal bovine calf serum (Gemini Bioproducts, Inc., Carlsbad, Calif.) and 2 mm glutamine, and 1% antibiotic-antimycotic in a humidified atmosphere of 95% air and 5% CO₂ at 37° C. Briefly, HT-29 cells are plated at a density of 500 cells/well in 96 well microtiter plates and incubated for 24 hours at 37° C. prior to the addition of compound. Each determination of cell number involves six replicates. After six days in culture, the cells are fixed by the addition of cold trichloroacetic acid to a final concentration of 10% and protein levels are measured using the sulforhodamine B (SRB) colorimetric protein stain assay as previously described by Skehan, P., Storeng, R., Scudiero, D., Monks, A., McMahon, J., Vistica, D., Warren, J. T., Bokesch, H., Kenney, S., and Boyd, M. R., “New Colorimetric Assay For Anticancer-Drug Screening,” J. Natl. Cancer Inst. 82: 1107-1112, 1990, which is incorporated herein by reference.

In addition to the SRB assay described above, a number of other methods are available to measure growth inhibition and could be substituted for the SRB assay. These methods include counting viable cells following trypan blue staining, labeling cells capable of DNA synthesis with BrdU or radiolabeled thymidine, neutral red staining of viable cells, or MTT staining of viable cells.

Significant tumor cell growth inhibition greater than about 50% at a therapeutically effective dose is indicative that the composition is useful for treating neoplastic lesions.

### Example 3

### Mammary Gland Organ Culture Model Tests

Compositions can also be tested for antineoplastic activity by their ability to inhibit the incidence of pre-neoplastic lesions in a mammary gland organ culture system. This mouse mammary gland organ culture technique has been successfully used by other investigators to study the effects of known antineoplastic agents such as certain NSAIDs, retinoids, tamoxifen, selenium, and certain natural products.

For example, female BALB/c mice can be treated with a combination of estradiol and progesterone daily, in order to prime the glands to be responsive to hormones in vitro. The animals are sacrificed, and thoracic mammary glands are excised aseptically and incubated for ten days in growth media supplemented with insulin, prolactin, hydrocortisone, and aldosterone. DMBA (7,12 dimethylbenz(a)anthracene) is added to medium to induce the formation of premalignant lesions. Fully developed glands are then deprived of prolactin, hydrocortisone, and aldosterone, resulting in the regression of the glands but not the pre-malignant lesions.

The test composition is dissolved in DMSO and added to the culture media for the duration of the culture period. At the end of the culture period, the glands are fixed in 10% formalin, stained with alum carmine, and mounted on glass slides. The incidence of forming mammary lesions is the ratio of the glands with mammary lesions to glands without lesions. The incidence of mammary lesions in test composition treated glands is compared with that of the untreated glands.

The extent of the area occupied by the mammary lesions can be quantitated by projecting an image of the gland onto a digitation pad. The area covered by the gland is traced on the pad and considered as 100% of the area. The space covered by each of the non-regressed structures is also outlined on the digitization pad and quantitated by the computer.

1. A method for treating a neoplasia, the method comprising:
(a) diagnosing a subject in need of treatment for a neoplasia; and
(b) administering to the subject a cyclooxygenase-2 selective inhibitor or an isomer, a pharmaceutically acceptable salt, ester, or prodrug thereof and a serotonin modulating agent or an isomer, ester, pharmaceutically acceptable salt or a prodrug thereof.

2. The method of claim 1 wherein the cyclooxygenase-2 selective inhibitor has a selectivity ratio of COX-1 IC₅₀ to COX-2 IC₅₀ not less than about 50.

3. The method of claim 1 wherein the cyclooxygenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, cimicoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S-6,8-dichloro-2-(trifluoromethyl)-2H-1-benzopyran-3-carboxylic acid, or an isomer, ester, a pharmaceutically acceptable salt or a prodrug thereof.

5. The method of claim 1 wherein the serotonin modulating agent is selected from the group consisting of
citalopram,
fluoxetine,
fluvoxamine,
paroxetine,
escitalopram oxalate,
sertraline,
palonosetron,
3-[4-(4-chlorophenyl)piperazin-1-yl]-1,1-diphenyl-2-propanol,
4-[3-[t-butylamino]-2-hydroxypropoxy]-1H-indole-2-carbonitrile,
4-(5H-dibenzo[a,d]cyclohepten-5-ylidene)-1-methylpiperidine,
3-(4-allylpiperazin-1-yl)-2-quinoxalinecarbonitrile maleate,
tropanyl 3,5-dichlorobenzoate,
1-methyl-1H-indole-3-carboxylic acid,
3-(piperidin-1-yl)propyl-4-amino-5-chloro-2-methoxybenzoate,
metergoline phenylmethyl ester,
6-chloro-2-[piperidinyl-4-thio]pyridine,
α-methyl-5-(2-thienylmethoxy)-1H-indole-3-ethanamine,
1-(3-chlorophenyl)piperazine,
2-methyl-5-hydroxytryptamine hydrochloride,
2-[1-(4-piperonyl)piperazinyl]benzothiazole,
5-carboxamidotryptamine maleate,
2-methyl-5-hydroxytryptamine hydrochloride,
N-acetyltryptamine,
4-[4-(2-phenylethyl)-1-piperazinyl]-benzo[b]thiophene-2-nitrile, and
4-(1-piperazinyl)benzo[b]thiophene-2-(N-methyl)carboxamide,
or an isomer, a pharmaceutically acceptable salt, ester, or prodrug thereof.

6. The method of claim 4 wherein the serotonin modulating agent is selected from the group consisting of
citalopram,
fluoxetine,
fluvoxamine,
paroxetine,
escitalopram oxalate,
sertraline,
palonosetron,
3-[4-(4-chlorophenyl)piperazin-1-yl]-1,1-diphenyl-2-propanol,
4-[3-[t-butylamino]-2-hydroxypropoxy]-1H-indole-2-carbonitrile,
4-(5H-dibenzo[a,d]cyclohepten-5-ylidene)-1-methylpiperidine,
3-(4-allylpiperazin-1-yl)-2-quinoxalinecarbonitrile maleate,
tropanyl 3,5-dichlorobenzoate,
1-methyl-1H-indole-3-carboxylic acid,
3-(piperidin-1-yl)propyl-4-amino-5-chloro-2-methoxybenzoate,
metergoline phenylmethyl ester,
6-chloro-2-[piperidinyl-4-thio]pyridine,
α-methyl-5-(2-thienylmethoxy)-1H-indole-3-ethanamine,
1-(3-chlorophenyl)piperazine,
2-methyl-5-hydroxytryptamine hydrochloride,
2-[1-(4-piperonyl)piperazinyl]benzothiazole,
5-carboxamidotryptamine maleate,
2-methyl-5-hydroxytryptamine hydrochloride,
N-acetyltryptamine,
4-[4-(2-phenylethyl)-1-piperazinyl]-benzo[b]thiophene-2-nitrile, and
4-(1-piperazinyl)benzo[b]thiophene-2-(N-methyl)carboxamide,
or an isomer, a pharmaceutically acceptable salt, ester, or prodrug thereof.

7. The method of claim 1 wherein the cyclooxygenase-2 selective inhibitor and the serotonin modulating agent are administered substantially simultaneously.

8. The method of claim 1 wherein the cyclooxygenase-2 selective inhibitor and the serotonin modulating agent are administered sequentially.

9. The method of claim 1 wherein the cyclooxygenase-2 selective inhibitor is administered to the subject in an amount of about 0.1 to about 20 mg/kg body weight per day.

10. The method of claim 1 wherein the serotonin modulating agent is administered to the subject in an amount of about 10 to about 500 milligrams per day.

11. A method for treating a neoplasia, the method comprising:
(a) diagnosing a subject in need of treatment for a neoplasia; and
(b) administering to the subject a serotonin modulating agent or an isomer, a pharmaceutically acceptable salt, ester, or prodrug thereof, and a cyclooxygenase-2 selective inhibitor or an isomer, ester, a pharmaceutically acceptable salt, or a prodrug thereof, wherein the cyclooxygenase-2 selective inhibitor is a chromene compound, the chromene compound comprising a benzothiopyran, a dihydroquinoline or a dihydronaphthalene.

12. The method of claim 11 wherein the cyclooxygenase-2 selective inhibitor has a selectivity ratio of COX-1 IC₅₀ to COX-2 IC₅₀ not less than about 50.

13. The method of claim 11 wherein the cyclooxygenase-2 selective inhibitor has a selectivity ratio of COX-1 IC₅₀ to COX-2 IC₅₀ not less than about 100.

14. The method of claim 11 wherein the cyclooxygenase-2 selective inhibitor or an isomer, ester, a pharmaceutically acceptable salt, or a prodrug thereof is a compound having the formula: 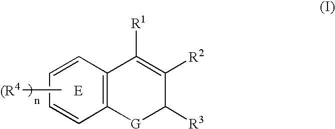
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

15. The method of claim 11 wherein the cyclooxygenase-2 selective inhibitor is (S)-6,8-dichloro-2-(trifluoromethyl)-2H-1-benzopyran-3-carboxylic acid or an isomer, a pharmaceutically acceptable salt, ester, or prodrug thereof.

16. The method of claim 11 wherein the serotonin modulating agent is selected from the group consisting of
citalopram,
fluoxetine,
fluvoxamine,
paroxetine,
escitalopram oxalate,
sertraline,
palonosetron,
3-[4-(4-chlorophenyl)piperazin-1-yl]-1,1-diphenyl-2-propanol,
4-[3-[t-butylamino]-2-hydroxypropoxy]-1H-indole-2-carbonitrile,
4-(5H-dibenzo[a,d]cyclohepten-5-ylidene)-1-methylpiperidine,
3-(4-allylpiperazin-1-yl)-2-quinoxalinecarbonitrile maleate,
tropanyl 3,5-dichlorobenzoate,
1-methyl-1H-indole-3-carboxylic acid,
3-(piperidin-1-yl)propyl-4-amino-5-chloro-2-methoxybenzoate,
metergoline phenylmethyl ester,
6-chloro-2-[piperidinyl-4-thio]pyridine,
α-methyl-5-(2-thienylmethoxy)-1H-indole-3-ethanamine,
1-(3-chlorophenyl)piperazine,
2-methyl-5-hydroxytryptamine hydrochloride,
2-[1-(4-piperonyl)piperazinyl]benzothiazole,
5-carboxamidotryptamine maleate,
2-methyl-5-hydroxytryptamine hydrochloride,
N-acetyltryptamine,
4-[4-(2-phenylethyl)-1-piperazinyl]-benzo[b]thiophene-2-nitrile, and
4-(1-piperazinyl)benzo[b]thiophene-2-(N-methyl)carboxamide,
or an isomer, a pharmaceutically acceptable salt, ester, or prodrug thereof.

17. A method for treating a neoplasia, the method comprising:
(a) diagnosing a subject in need of treatment for a neoplasia; and
(b) administering to the subject a serotonin modulating agent or an isomer, a pharmaceutically acceptable salt, ester, or prodrug thereof, and a cyclooxygenase-2 selective inhibitor or an isomer, ester, a pharmaceutically acceptable salt, or a prodrug thereof, wherein the cyclooxygenase-2 selective inhibitor is a tricyclic compound, the tricyclic compound containing a benzenesulfonamide or methylsulfonylbenzene moiety.

18. The method of claim 17 wherein the cyclooxygenase-2 selective inhibitor has a selectivity ratio of COX-1 IC₅₀ to COX-2 IC₅₀ not less than about 50.

19. The method of claim 17 wherein the cyclooxygenase-2 selective inhibitor has a selectivity ratio of COX-1 IC₅₀ to COX-2 IC₅₀ not less than about 100.

20. The method of claim 17 wherein the cyclooxygenase-2 selective inhibitor or an isomer, ester, a pharmaceutically acceptable salt, or a prodrug thereof is a compound of the formula: 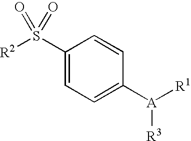
wherein:
A is selected from the group consisting of a partially unsaturated or unsaturated heterocyclyl ring and a partially unsaturated or unsaturated carbocyclic ring;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

21. The method of claim 17 wherein the cyclooxygenase-2 selective inhibitor is selected from the group consisting of celecoxib, cimicoxib, valdecoxib, parecoxib, deracoxib, rofecoxib, etoricoxib, and 2-(3,4-difluorophenyl)-4-(3-hydroxy-3-methylbutoxy)-5-[4-(methylsulfonyl)phenyl]-3(2H)-pyridazinone or an isomer, a pharmaceutically acceptable salt, ester, or prodrug thereof.

22. The method of claim 17 wherein the serotonin modulating agent is selected from the group consisting
citalopram,
fluoxetine,
fluvoxamine,
paroxetine,
escitalopram oxalate,
sertraline,
palonosetron,
3-[4-(4-chlorophenyl)piperazin-1-yl]-1,1-diphenyl-2-propanol,
4-[3-[t-butylamino]-2-hydroxypropoxy]-1H-indole-2-carbonitrile,
4-(5H-dibenzo[a,d]cyclohepten-5-ylidene)-1-methylpiperidine,
3-(4-allylpiperazin-1-yl)-2-quinoxalinecarbonitrile maleate,
tropanyl 3,5-dichlorobenzoate,
1-methyl-1H-indole-3-carboxylic acid,
3-(piperidin-1-yl)propyl4-amino-5-chloro-2-methoxybenzoate,
metergoline phenylmethyl ester,
6-chloro-2-[piperidinyl-4-thio]pyridine,
α-methyl-5-(2-thienylmethoxy)-1H-indole-3-ethanamine,
1-(3-chlorophenyl)piperazine,
2-methyl-5-hydroxytryptamine hydrochloride,
2-[1-(4-piperonyl)piperazinyl]benzothiazole,
5-carboxamidotryptamine maleate,
2-methyl-5-hydroxytryptamine hydrochloride,
N-acetyltryptamine,
4-[4-(2-phenylethyl)-1-piperazinyl]-benzo[b]thiophene-2-nitrile, and
4-(1-piperazinyl)benzo[b]thiophene-2-(N-methyl)carboxamide,
or an isomer, a pharmaceutically acceptable salt, ester, or prodrug thereof.

23. A method for treating a neoplasia, the method comprising:
(a) diagnosing a subject in need of treatment for a neoplasia; and
(b) administering to the subject a serotonin modulating agent or an isomer, a pharmaceutically acceptable salt, ester, or prodrug thereof, and a cyclooxygenase-2 selective inhibitor or an isomer, ester, a pharmaceutically acceptable salt, or a prodrug thereof, wherein the cyclooxygenase-2 selective inhibitor is a phenyl acetic acid compound.

24. The method of claim 23 wherein the cyclooxygenase-2 selective inhibitor has a selectivity ratio of COX-1 IC₅₀ to COX-2 IC₅₀ not less than about 50.

25. The method of claim 23 wherein the cyclooxygenase-2 selective inhibitor has a selectivity ratio of COX-1 IC₅₀ to COX-2 IC₅₀ not less than about 100.

26. The method of claim 23 wherein the cyclooxygenase-2 selective inhibitor is a compound having the formula: 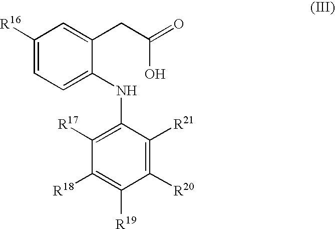
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl; provided, however, that each of R¹⁷, R¹⁸, R²⁰ and R²¹ is not fluoro when R¹⁶ is ethyl and R¹⁹ is H.

27. The method of claim 26 wherein R¹⁶ is ethyl, R¹⁷ and R¹⁹ are chloro, R¹⁸ and R²⁰ are hydrogen; and R²¹ is methyl.

28. The method of claim 23 wherein the serotonin modulating agent is selected from the group consisting of
citalopram,
fluoxetine,
fluvoxamine,
paroxetine,
escitalopram oxalate,
sertraline,
palonosetron,
3-[4-(4-chlorophenyl)piperazin-1-yl]-1,1-diphenyl-2-propanol,
4-[3-[t-butylamino]-2-hydroxypropoxy]-1H-indole-2-carbonitrile,
4-(5H-dibenzo[a,d]cyclohepten-5-ylidene)-1-methylpiperidine,
3-(4-allylpiperazin-1-yl)-2-quinoxalinecarbonitrile maleate,
tropanyl 3,5-dichlorobenzoate,
1-methyl-1H-indole-3-carboxylic acid,
3-(piperidin-1-yl)propyl-4-amino-5-chloro-2-methoxybenzoate,
metergoline phenylmethyl ester,
6-chloro-2-[piperidinyl-4-thio]pyridine,
α-methyl-5-(2-thienylmethoxy)-1H-indole-3-ethanamine,
1-(3-chlorophenyl)piperazine,
2-methyl-5-hydroxytryptamine hydrochloride,
2-[1-(4-piperonyl)piperazinyl]benzothiazole,
5-carboxamidotryptamine maleate,
2-methyl-5-hydroxytryptamine hydrochloride,
N-acetyltryptamine,
4-[4-(2-phenylethyl)-1-piperazinyl]-benzo[b]thiophene-2-nitrile, and
4-(1-piperazinyl)benzo[b]thiophene-2-(N-methyl)carboxamide,
or an isomer, a pharmaceutically acceptable salt, ester, or prodrug thereof.

29. A method for treating a neoplasia, the method comprising:
(a) diagnosing a subject in need of treatment for a neoplasia; and
(b) administering to the subject a cyclooxygenase-2 selective inhibitor selected from the group consisting of celecoxib, cimicoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid, or an isomer, a pharmaceutically acceptable salt, ester, or prodrug thereof, and
a serotonin modulating agent selected from the group consisting of
citalopram,
fluoxetine,
fluvoxamine,
paroxetine,
escitalopram oxalate,
sertraline,
palonosetron,
3-[4-(4-chlorophenyl)piperazin-1-yl]-1,1-diphenyl-2-propanol,
4-[3-[t-butylamino]-2-hydroxypropoxy]-1H-indole-2-carbonitrile,
4-(5H-dibenzo[a,d]cyclohepten-5-ylidene)-1-methylpiperidine,
3-(4-allylpiperazin-1-yl)-2-quinoxalinecarbonitrile maleate,
tropanyl 3,5-dichlorobenzoate,
1-methyl-1H-indole-3-carboxylic acid,
3-(piperidin-1-yl)propyl-4-amino-5-chloro-2-methoxybenzoate,
metergoline phenylmethyl ester,
6-chloro-2-[piperidinyl-4-thio]pyridine,
α-methyl-5-(2-thienylmethoxy)-1H-indole-3-ethanamine,
1-(3-chlorophenyl)piperazine,
2-methyl-5-hydroxytryptamine hydrochloride,
2-[1-(4-piperonyl)piperazinyl]benzothiazole,
5-carboxamidotryptamine maleate,
2-methyl-5-hydroxytryptamine hydrochloride,
N-acetyltryptamine,
4-[4-(2-phenylethyl)-1-piperazinyl]-benzo[b]thiophene-2-nitrile, and
4-(1-piperazinyl )benzo[b]thiophene-2-(N-methyl)carboxamide,
or an isomer, a pharmaceutically acceptable salt, ester, or prodrug thereof.

30. The method of claim 29 wherein the cyclooxygenase-2 selective inhibitor and the serotonin modulating agent are combined and administered in the same dose.

31. The method of claim 29 wherein the cyclooxygenase-2 selective inhibitor and the serotonin modulating agent are administered in separate doses.
